(19) United States
(12) Patent Application Publication
(10) Pub. No.: US 2018/0168949 A1
(43) Pub. Date: Jun. 21, 2018

(54) BAG ON VALVE OR DIP-TUBE CONTAINER FOR A PRESSURIZED PERSONAL CARE PRODUCT

(71) Applicant: The Procter & Gamble Company, Cincinnati, OH (US)

(72) Inventors: Robert Wayne GLENN, JR., Liberty Twp., OH (US); Dariush Hosseinpour, Mason, OH (US); Toshiyuki Iwata, Singapore (SG); Kathleen Mary Kaufman, Cincinnati, OH (US); Kevin Lee Doyle, Fairfield, OH (US)

(21) Appl. No.: 15/843,146

(22) Filed: Dec. 15, 2017

Related U.S. Application Data
(60) Provisional application No. 62/435,271, filed on Dec. 16, 2016.

(51) Int. Cl. A61K 8/04; A61Q 5/02; A61Q 5/12; A61K 8/34; A61K 8/89; B65D 83/32; B65D 83/62

(52) U.S. Cl. CPC A61K 8/046; A61Q 5/02; A61Q 5/12; A61K 8/342; A61K 2800/21; B65D 83/32; B65D 83/62; A61K 2800/87; A61K 8/89

(57) ABSTRACT

Described herein is an aerosol dispenser including a pressurized personal care product in a bag on valve or dip-tube container. The pressurized personal care product includes from about 2% to about 10% propellant, from about 0.5% to about 5% perfume, from about 1% to about 14% nonionic emulsifier, less than 5% cationic surfactant, from about 3% to about 17% silicone, and less than 7.5% high melting point fatty compound. The pressurized personal care composition has a liquid phase viscosity of from about 1 centipoise to about 15,000 centipoise. The pressurized hair care composition is rinse-off.

### FIELD OF THE INVENTION

Described herein is a pressurized personal care product in an aerosol bag on valve container.

### BACKGROUND OF THE INVENTION

Today's hair conditioners almost universally comprise high levels of high melting point fatty compounds, the most common of which are C16 to C18 fatty alcohols. These high melting point fatty compounds are employed as structuring agents wherein they are combined with one or more surfactants and an aqueous carrier to form a gel network. The gel network provides a viscous and high yield point rheology which facilitates the dispensing of the conditioner from a bottle or tube and the subsequent distribution and spreading of the product through the hair by the consumer. The gel network structuring also enables incorporation of silicones, perfumes and oils in the form of an oil-in-water emulsion that is shelf stable. These silicones and oils are intended to be deposited on the hair to provide the primary hair conditioning benefits including wet and dry combing friction reduction and hair manageability etc.

However, today's gel network hair conditioners lead to excessive co-deposits of the high melting point fatty compound on the hair over multiple cycles. Additionally, the deposited high melting point fatty compounds build-up on hair over multiple cycles and lead to significant waxy build-up on hair and hair weigh down. Indeed, one of the major consumer complaints with hair conditioners is waxy residue which makes hair look greasy or feel heavy. Many current gel network hair conditioners deposit up to 10 times more high melting point fatty compounds (fatty alcohols) than silicone or oil after approximately 10 treatment cycles in technical testing. While not being bound to theory, this is hypothesized to be due to the greater concentration of high melting point weight fatty compounds in the product relative to the silicone or oil. Importantly, such a high level of melting point fatty compounds (fatty alcohols) is required to produce a shelf stable gel network with sufficient structuring for consumer acceptable viscosity and rheology.

Described herein is a pressurized hair care composition that enables new product opportunities and consumer benefits by addressing the current disadvantages associated with gel network conditioners. It has been found that concentrated and low viscosity silicone nanoemulsion hair conditioner compositions can be delivered to the hair in foamed form. These new concentrated silicone nanoemulsion compositions enable sufficient dosage from a low density and lower dosage foam delivery form while also minimizing the need for high melting point fatty compounds or other “insoluble” structurants that can lead to significant co-deposits, build-up and weigh down of hair. The net result has been a step change improvement in silicone deposition purity versus today's rinse-off products and an improvement in technical performance benefits from such a pure and transparent deposited silicone layer. These benefits include multicycle hair conditioning without hair weigh down, durable conditioning, fizz control, targeted deposition onto damaged hair, reduced hair dye fade, and increased color vibrancy.

Nanoemulsion technology development is hindered by complex stability issues that emerge when droplet sizes are driven to the nanoscale. This is especially problematic in the presence of higher levels of perfume oils required for such a concentrated product. The pressurized hair care composition described herein is therefore also focused on improved stability.

### SUMMARY OF THE INVENTION

Described herein is an aerosol dispenser comprising a pressurized hair care composition, the pressurized hair care composition comprising: (a) from about 3% to about 17% of one or more silicones, by weight of the pressurized hair care composition, wherein the particle size of the one or more silicones is from about 1 nm to about 500 nm; (b) less than 7.5% high melting point fatty compounds, by weight of the pressurized hair care composition; (c) from about 2% to about 10% propellant, by weight of the pressurized hair care composition; (d) less than 5% cationic surfactant, by weight of the pressurized hair care composition; (e) from about 0.5% to about 5% perfume, by weight of the pressurized hair care composition; (f) from about 1% to about 14% of a nonionic emulsifier; and (g) from about 55% to about 87% water, by weight of the pressurized hair care composition; wherein the pressurized hair care composition has a liquid phase viscosity of from about 1 centipoise to about 15,000 centipoise before addition of the propellant; wherein the pressurized hair care composition has a high melting point fatty compound to silicone weight ratio of from about 0 to about 50:50; wherein the pressurized hair care composition has a silicone to perfume weight ratio of from about about 50:50 to about 95:5; wherein the aerosol dispenser dispenses a foam having a density of from about 0.025 g/cm³ to about 0.40 g/cm³ when dispensed from the aerosol dispenser; and wherein the pressurized hair care composition is rinse-off.

Also described herein is a method of conditioning the hair, the method comprising: (a) providing a hair care composition, wherein the hair care composition comprises: (i) from about 3% to about 18% of one or more silicones, by weight of the hair care composition, wherein the particle size of the one or more silicones is from about 1 nm to about 500 nm; (ii) less than 8% high melting point fatty compound, by weight of the hair care composition; (iii) less than 5% cationic surfactant, by weight of the hair care composition; (iv) from about 0.5% to about 5% perfume, by weight of the hair care composition; (v) from about 1% to about 15% of a nonionic emulsifier, by weight of the hair care composition; and (vi) from about 60% to about 90% water, by weight of the hair care composition; wherein the hair care composition has a liquid phase viscosity of from about 1 centipoise to about 15,000 centipoise; wherein the hair care composition has a high melting point fatty compound to silicone weight ratio of from about 0 to about 50:50; and wherein the hair care composition has a silicone to perfume weight ratio of from about 50:50 to about 95:5; (b) adding a propellant to the hair care composition at a concentrated hair care composition to propellant weight ratio of from about 85:15 to about 98:2 to create a pressurized hair care composition; (c) dispensing the pressurized hair care composition from an aerosol dispenser as a foam; (d) applying the foam to the hair; and (e) rinsing the foam from the hair; wherein the foam has a density of from about 0.025 g/cm³ to about 0.40 g/cm³ when dispensed from the aerosol dispenser.

### BRIEF DESCRIPTION OF THE DRAWINGS

While the specification concludes with claims, it is believed that the same will be better understood from the following description taken in conjunction with the accompanying drawings in which:

FIG. 1 is a comparative SEM image of a hair treated with a Pantene Clarifying Shampoo plus a Pantene Anti-breakage Conditioner;

FIG. 2 is an SEM image of a hair treated with a Pantene Clarifying Shampoo plus the pressurized hair care composition of Example 1 from Table 2;

FIG. 3 is an SEM image of a hair treated with a Pantene Clarifying Shampoo plus the pressurized hair care composition of Example 2 from Table 2;

FIG. 4 is an SEM image of a hair treated with a Pantene Clarifying Shampoo plus the pressurized hair care composition of Example 3 from Table 2;

FIG. 5 is an SEM image of a hair treated with a Pantene Clarifying plus the pressurized hair care composition of Example 4 from Table 2;

FIG. 6 is an SEM image of a hair treated with Foam Shampoo 1 from Table 1 plus the pressurized hair care composition of Example 2 from Table 2; and

FIG. 7 is an SEM image of a hair treated with Foam Shampoo 2 from Table 1 plus the pressurized hair care composition of Example 2 from Table 2.

### DETAILED DESCRIPTION OF THE INVENTION

While the specification concludes with claims particularly pointing out and distinctly claiming the invention, it is believed that the present invention will be better understood from the following description.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “M.Wt.” refers to the weight average molecular weight unless otherwise stated.

As used herein, the terms “include,” “includes,” and “including,” are meant to be non-limiting and are understood to mean “comprise,” “comprises,” and “comprising,” respectively.

As used herein, the term “nanoemulsion” means an oil-in-water (o/w) emulsion with an average particle size ranging from about 1 nm to about 100 nm. The particle size referred to herein is z-average measured by dynamic light scattering. The nanoemulsion described herein may be prepared by the following methods: (1) mechanically breaking down the emulsion droplet size; (2) spontaneously forming the emulsion (may be referred to as a microemulsion in the literature); and (3) using emulsion polymerization to achieve average particle size in the target range described herein.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The method of conditioning the hair described herein comprises (a) providing a hair care composition; and (b) adding a propellant to the hair care composition to create a pressurized hair care composition. The hair care composition and/or the pressurized hair care composition can comprise the following:

A. Silicone Deposition Purity

The method of treating hair comprises dispensing the pressurized hair care composition described herein from the aerosol dispenser as a dosage of foam. The foam may comprise a silicone deposition purity of from about 40% to about 100%, alternatively from about 50% to about 100%, alternatively from about 55% to about 100%, alternatively from about 60% to about 100%, alternatively from about 65% to about 100%, alternatively from about 70% to about 100%, and alternatively from about 80% to about 100%, after applying the foam to hair which has previously been cleaned with a clarifying shampoo free of waxes and hydrophobic conditioning agents; rinsing the foam from the hair; and drying the hair. The foam may comprise a silicone deposition purity of from 50% to about 90%, alternatively from about 55% to about 85%, and alternatively from about 60% to about 80%, after applying the foam to hair which has previously been cleaned with a clarifying shampoo free of waxes and hydrophobic conditioning agents; and rinsing the foam from the hair; and drying the hair.The foam may comprise a silicone deposition purity from about 50% to about 100%, alternatively from about 55% to about 100%, alternatively from about 60% to about 100%, alternatively from about 65% to about 100%, alternatively from about 68% to about 99%, and alternatively from about 75% to about 95% after applying the foam to general population hair which has previously been cleaned with a clarifying shampoo free of waxes and hydrophobic conditioning agents; and rinsing the foam from the hair; and drying the hair.

Silicone Deposition Purity is determined by the ratio of silicone deposited per weight of hair to the total deposition of other ingredients per weight of hair. Silicone is determined by either extraction or digestion of the hair followed by an analysis with a quantitative elemental technique such as ICP for total silicon and converting to silicone based on the % of silicon in the silicone by weight. The total deposition may be determined by the sum of separate deposition measurements or by a Single Inclusive Measurement of total deposition. The separate deposition measurements may include but are not limited to: fatty alcohols, EGDS, quaternized agents and silicone. Typically these measurements involve extracting the hair then separating the ingredients of interest with chromatography and quantifying with an externally calibration based on test solution concentration. The Single Inclusive Measurement of total deposition is gravimetric.

The hair is thoroughly extracted and the residue determined by weighing the dissolved residue in the extract after evaporating the solvent. This residue contains both deposited ingredients and naturally occurring extractable compounds from the hair (primarily lipids). The naturally occurring extractable compounds are quantified and subtracted from the total. These include: fatty acids, squalene, cholesterol, ceramides, wax esters, triglycerides and sterol esters. The method of quantitation is similar to the deposition measurements. Other supporting evidence of Deposition Purity may include spectroscopic or topography mapping of the hair surface.

B. Silicone to Fatty Alcohol Deposition Weight Ratio

The foam may comprise a silicone to fatty alcohol deposition weight ratio of from about 50:50 to about 100:0, alternatively from about 55:45 to about 100:0, alternatively from about 60:40 to about 100:0, alternatively from about 65:35 to about 100:0, alternatively from about 70:30 to about 100:0, alternatively from about 55:45 to about 95:5, alternatively from about 60:40 to about 90:10, alternatively from about 65:35 to about 85:15, and alternatively from about 70:30 to about 80:20 after (1) applying the foam to hair which has previously been cleaned with a clarifying shampoo free of waxes and hydrophobic conditioning agents; (2) rinsing the foam from the hair; and (3) drying the hair.

The foam may comprise a silicone to fatty alcohol weight ratio of from about 0 to about 50:50, alternatively from about 0 to about 45:55, alternatively from about 0 to about 40:60, alternatively from about 0 to about 35:65, alternatively from about 0 to about 30:70, and alternatively from about 0 to about 33:67 after (1) applying the foam to hair which has previously been cleaned with a clarifying shampoo free of waxes and hydrophobic conditioning agents; (2) rinsing the foam from the hair; and (3) drying the hair. The foam may comprise a silicone to fatty alcohol deposition weight ratio of from about 5:95 to about 45:55, alternatively from about 10:90 to about 40:60, alternatively from about 15:85 to about 35:65, and alternatively from about 20:80 to about 30:70 after (1) applying the foam to hair which has previously been cleaned with a clarifying shampoo free of waxes and hydrophobic conditioning agents; (2) rinsing the foam from the hair; and (3) drying the hair.

The weight of the silicone deposited onto the hair can be determined by digestion of the hair followed by an analysis with a quantitative elemental technique such as ICP for total silicon and converting to μg silicone/g of hair (ppm) based on the % of silicon in the silicone.

The weight of the fatty alcohol deposited onto the hair can be determined by extraction followed by quantitation via capillary gas chromatography. The resulting peaks are integrated and μg fatty alcohol/g of hair (ppm) is calculated using the internal standard mode.

The fatty alcohol deposition can be greater than or equal to 0 and less than 1000 ppm, alternatively greater than or equal to 0 and less than 950 ppm, alternatively greater than or equal to 0 and less than 900 ppm, alternatively greater than or equal to 0 and less than 850, alternatively greater than or equal to 0 and less than 800, alternatively greater than or equal to 0 and less than 750 ppm, alternatively greater than or equal to 0 and less than 700 ppm, alternatively greater than or equal to 0 and less than 700, and alternatively greater than or equal to 0 and less 650 ppm.

C. Silicones

The hair care composition may comprise from about 3% to about 18%, alternatively from about 4% to about 16%, alternatively from about 5% to about 14%, alternatively from about 6% to about 12%, alternatively from about 6% to about 10%, and alternatively from about 4% to about 7% of one or more silicones by weight of the hair care composition.

The pressurized hair care composition may comprise from about 3% to about 17%, alternatively from about 4% to about 15%, alternatively from about 5% to about 13%, alternatively from about 6% to about 11%, alternatively from about 6% to about 10%, and alternatively from about 4% to about 7% of one or more silicones, by weight of the pressurized hair care composition.

The particle size of the one or more silicones in the hair care composition can be from about 1 nm to about 100 nm, alternatively from about 5 nm to about 80 nm, alternatively from about 10 nm to about 60 nm, and alternatively from about 12 nm to about 50 nm. Alternatively, the particle size of the one or more silicones in the hair care composition can be from about 1 nm to about 500 nm, alternatively from about 5 nm to about 300 nm, alternatively from about 8 nm to about 200 nm, and alternatively from about 10 nm to about 100 nm.

The particle size of the one or more silicones can be measured by dynamic light scattering (DLS) using a measurement angle of 173° and the refractive index of the one or more silicones. A Malvern Zetasizer Nano ZEN3600 system using He-Ne laser 633 nm can be used for the measurement at 25° C. For each sample, three particle size measurements are taken and the Z-average value is reported as the particle size.

The one or more silicones may be in the form of a nanoemulsion. A nanoemulsion, as defined herein, is an emulsion wherein the particle size is below 100 nm. The nanoemulsion may comprise any silicone suitable for application to the skin and/or hair. From about 25% to about 100% of the total silicone in the hair care composition and/or the pressurized hair care composition can be in the form of a nanoemulsion, alternatively from about 50% to about 100% of the total silicone in the hair care composition and/or the pressurized hair care composition can be in the form of a nanoemulsion, and alternatively from about 75% to about 100% of the total silicone in the hair care composition and/or the pressurized hair care composition can be in the form of a nanoemulsion.

The one or more silicones may include in their molecular structure polar functional groups such as Si—OH (present in dimethiconols), primary amines, secondary amines, tertiary amines, and quaternary ammonium salts. The one or more silicones may be selected from the group consisting of aminosilicones, pendant quaternary ammonium silicones, terminal quaternary ammonium silicones, amino polyalkylene oxide silicones, quaternary ammonium polyalkylene oxide silicones, and amino morpholino silicones.

The one or more silicones can include one or more aminosilicones corresponding to formula (I):

R′ₐG₃₋ₐ-Si(OSiG₂)ₙ-(OSiG_{b}R′_{2-b})ₘ—O—SiG₃₋ₐ-R′ₐ (I)
in which:
G is chosen from a hydrogen atom, a phenyl group, OH group, and C₁-C₈ alkyl groups, for example methyl,
a is an integer ranging from 0 to 3, and alternatively a is 0,
b is chosen from 0 and 1, and alternatively b is 1,
m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10;
R′ is a monovalent group of formula —C_{q}H_{2q}L in which q is a number from 2 to 8 and L is an optionally quaternized amine group chosen from the groups:
—NR″—CH₂—CH₂—N′(R¹)₂,
—N(R″)₂,
—N⁺(R″)₃A⁻,
—N⁺H(R″)₂A⁻,
—N⁺H₂(R″)A⁻, and
—N(R″)—CH₂—CH₂—N⁺R″H₂A⁻,
   in which R″ can be chosen from a hydrogen atom, phenyl groups, benzyl groups, and saturated monovalent hydrocarbon-based groups, such as for example an alkyl group comprising from 1 to 20 carbon atoms, and A⁻ is chosen from halide ions such as, for example, fluoride, chloride, bromide and iodide.

The one or more silicones may include those corresponding to formula (1) wherein a=0, G=methyl, m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10; and L is —N(CH₃)₂ or —NH₂, alternatively —NH₂.

The one or more silicones can include pendant quaternary ammonium silicones of formula (II):

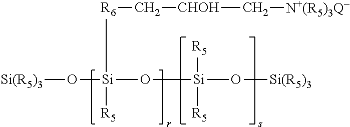

in which:
R₅ is chosen from monovalent hydrocarbon-based groups comprising from 1 to 18 carbon atoms, such as C₁-C₁₈ alkyl groups and C₂-C₁₈ alkenyl groups, for example methyl;
R₆ is chosen from divalent hydrocarbon-based groups, such as divalent C₁-C₁₈ alkylene groups and divalent C₁-C₁₈ alkylenoxy groups, for example C₁-C₈ alkylenoxy groups, wherein said R₆ is bonded to the Si by way of an SiC bond;
Q⁻ is an anion that can be for example chosen from halide ions, such as chloride, and organic acid salts (such as acetate);
r is an average statistical value ranging from 2 to 20, such as from 2 to 8;
s is an average statistical value ranging from 20 to 200, such as from 20 to 50.

Such aminosilicones are described more particularly in U.S. Pat. No. 4,185,087, the disclosure of which is incorporated by reference herein.

A silicone which falls within this class is the silicone sold by the company Union Carbide under the name “Ucar Silicone ALE 56”.

Further examples of the one or more silicones include quaternary ammonium silicones of formula (III):

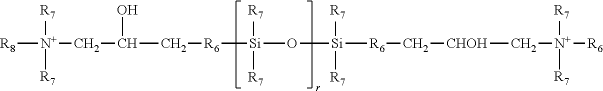

in which:
groups R₇, which may be identical or different, are each chosen from monovalent hydrocarbon-based groups comprising from 1 to 18 carbon atoms, such as C₁-C₁₈ alkyl groups, for example methyl, C₂-C₁₈ alkenyl groups, and rings comprising 5 or 6 carbon atoms;
R₆ is chosen from divalent hydrocarbon-based groups, such as divalent C₁-C₁₈ alkylene groups and divalent C₁-C₁₈ alkylenoxy, for example C₁-C₈, group connected to the Si by an SiC bond;
R_{8,} which may be identical or different, represent a hydrogen atom, a monovalent hydrocarbon-based group comprising from 1 to 18 carbon atoms, and in particular a C₁-C₁₈ alkyl group, a C₂-C₁₈ alkenyl group or a group —R₆—NHCOR₇;
X⁻ is an anion such as a halide ion, in particular chloride, or an organic acid salt (acetate, etc.);
r represents an average statistical value from 2 to 200 and in particular from 5 to 100.
Such silicones are described, for example, in application EP-A-0 530 974, the disclosure of which is incorporated by reference herein.
Silicones falling within this class can be the silicones sold by the company Goldschmidt under the names Abil Quat 3270, Abil Quat 3272 and Abil Quat 3474.
Further examples of the one or more silicones include quaternary ammonium and polyalkylene oxide silicones wherein the quaternary nitrogen groups are located in the polysiloxane backbone, at the termini, or both.
Such silicones are described in PCT Publication No. WO 2002/010257, the disclosure of which is incorporated by reference herein.
Siliciones falling within this class include silicones sold by the company Momentive under the name Silsoft Q™.

The one or more silicones can include aminofunctional silicones having morpholino groups of formula (IV):

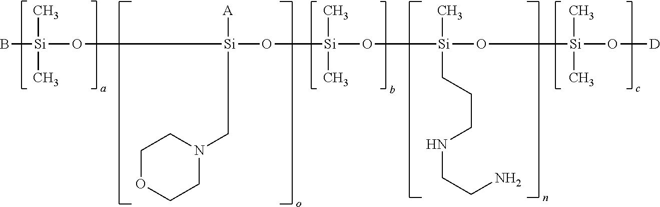

in which A denotes a structural unit (I), (II), or (III) bound via —O—

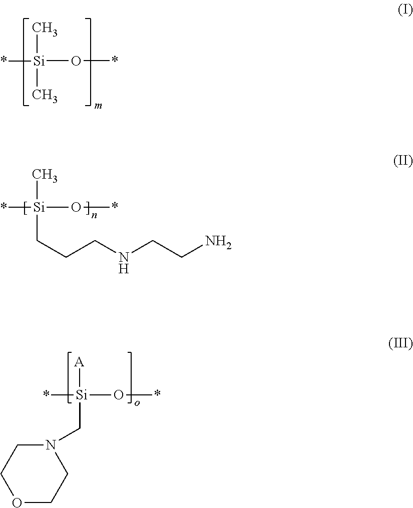

or an oligomeric or polymeric residue, bound via —O—, containing structural units of formulas (I), (II), or (III), or half of a connecting oxygen atom to a structural unit (III), or denotes —OH,
* denotes a bond to one of the structural units (I), (II), or (III), or denotes a terminal group B (Si-bound) or D (O-bound),
B denotes an —OH, —O—Si(CH₃)₃, —O—Si(CH₃)₂OH, —O—Si(CH₃)₂OCH₃ group,
D denotes an —H, —Si(CH₃)₃, —Si(CH₃)₂OH, —Si(CH₃)₂OCH₃ group,
a, b, and c denote integers between 0 and 1000, with the provision that a+b+c>0,
m, n, and o denote integers between 1 and 1000.

Aminofunctional silicones of this kind can bear the INCI name: Amodimethicone/Morpholinomethyl Silsesquioxane Copolymer. A particularly suitable amodimethicone is the product having the commercial name Wacker Belsil® ADM 8301E.

Examples of such silicones are available from the following suppliers:
offered by the company Dow Corning:
   Fluids: 2-8566, AP 6087, AP 6088, DC 8040 Fluid, fluid 8822A DC, DC 8803 & 8813 polymer, 7-6030, AP-8104, AP 8201;
   Emulsions: CE-8170 AF Micro Emulsion, 2-8177, 2-8194 Microemulsion, 9224 Emulsion, 939, 949, 959, DC 5-7113 Quat Microemulsion, DC 5-7070 Emulsion, DC CE-8810, CE 8401 Emulsion, CE 1619, Dow Corning Toray SS-3551, Dow Corning Toray SS-3552;
offered by the company Wacker:
   Wacker Belsil ADM 652, ADM 656, 1100, 1600, 1650 (fluids) ADM 6060 (linear amodimethicone) emulsion; ADM 6057 E (branched amodimethicone) emulsion; ADM 8020 VP (micro emulsion); SLM 28040 (micro emulsion);
offered by the Company Momentive:
   Silsoft 331, SF1708, SME 253 & 254 (emulsion), SM2125 (emulsion), SM 2658 (emulsion), Silsoft Q (emulsion)
offered by the company Shin-Etsu:
   KF-889, KF-8675, KF-8004, X-52-2265 (emulsion);
offered by the Company Siltech Silicones:
   Siltech E-2145, E-Siltech 2145-35;
offered by the company Evonik Industries:
   Abil T Quat 60th

Additional non-limiting examples of aminosilicones include the compounds having the following INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22, Quaternium-80, as well as Silicone Quaternium-2 Panthenol Succinate and Silicone Quaternium-16/Glycidyl Dimethicone Crosspolymer.

The aminosilicones can be supplied in the form of a nanoemulsion and include MEM 9049, MEM 8177, MEM 0959, MEM 8194, SME 253, and Silsoft Q.

The one or more silicones can include dimethicones, and/or dimethiconols. The dimethiconols are hydroxyl terminated dimethylsilicones represented by the general chemical formulas (V) and (VI):

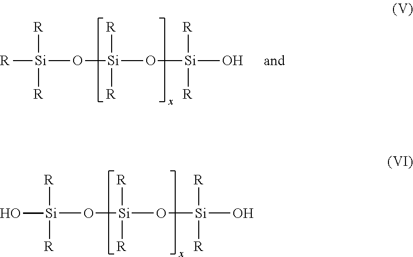

wherein R is an alkyl group (preferably R is methyl or ethyl, more preferably methyl) and x is an integer up to about 500, chosen to achieve the desired molecular weight. Commercial dimethiconols typically are sold as mixtures with dimethicone or cyclomethicone (e.g., Dow Coming® 1401, 1402, and 1403 fluids).

D. Nonionic Emulsifiers

The hair care composition can comprise from about 1% to about 15%, alternatively from about 2% to about 10%. and alternatively from about 2.5% to about 7.5% of a nonionic emulsifier, by weight of the hair care composition. The hair care composition may comprise from about 0% to about 20%, alternatively from about 0.01% to about 20%, alternatively from about 1% to about 15%, alternatively from about 2% to about 12%, alternatively from about 3% to about 10%, and alternatively from about 4% to about 8% of a nonionic emulsifier, by weight of the hair care composition.

The pressurized hair care composition can comprise from about 1% to about 14%, alternatively from about 2% to about 9.5%, and alternatively from about 2.5% to about 7.5% of a nonionic emulsifier, by weight of the pressurized hair care composition. The pressurized hair care composition can comprise from about 0% to about 19%, alternatively from about 0.01% to about 19%, alternatively from about 1% to about 14%, alternatively from about 2% to about 12%, alternatively from about 3% to about 10%, and alternatively from about 4% to about 8% of a nonionic emulsifier, by weight of the pressurized hair care composition.

Nonionic emulsifiers may be broadly defined as including compounds containing an alkylene oxide groups (hydrophilic in nature) with a hydrophobic compound, which may be aliphatic or alkyl aromatic in nature. Examples of nonionic emulsifiers include:

1. Alcohol ethoxylates which are condensation products of aliphatic alcohols having from about 8 to about 18 carbon atoms, in either straight chain or branched chain configuration, with from about 2 to about 35 moles of ethylene oxide, e.g., a coconut alcohol ethylene oxide condensate having from about 2 to about 30 moles of ethylene oxide per mole of coconut alcohol, the coconut alcohol fraction having from about 10 to about 14 carbon atom.

2. The polyethylene oxide condensates of alkyl phenols, e.g., the condensation products of the alkyl phenols having an alkyl group containing from about 6 to about 20 carbon atoms in either a straight chain or branched chain configuration, with ethylene oxide, the said ethylene oxide being present in amounts equal to from about 3 to about 60 moles of ethylene oxide per mole of alkyl phenol.

3. Those derived from the condensation of ethylene oxide with the product resulting from the reaction of propylene oxide and ethylene diamine products.

4. Long chain tertiary amine oxides such as those corresponding to the following general formula: R1 R2 R3 N→O wherein R₁ contains an alkyl, alkenyl or monohydroxy alkyl redical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals (the arrow in the formula represents a semipolar bond).

5. Long chain tertiary phosphine oxides corresponding to the following general formula: RR′R″P→O wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms. The arrow in the formula represents a semipolar bond.

6. Long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety.

7. Polysorbates, e.g., sucrose esters of fatty acids. Such materials are described in U.S. Pat. No. 3,480,616, e.g., sucrose cocoate (a mixture of sucrose esters of a coconut acid, consisting primarily of monoesters, and sold under the tradenames GRILLOTEN LSE 87K from RITA, and CRODESTA SL-40 from Croda).

8. Alkyl polysaccharide nonionic emulsifiers are disclosed in U.S. Pat. No. 4,565,647, Lienado, issued Jan. 21, 1986, having a hydrophobic group containing from about 6 to about 30 carbon atoms, preferably from about 10 to about 16 carbon atoms and a polysaccharide, e.g., a polyglycoside, hydrophilic group. The polysaccharide can contain from about 1.0 to about 10, alternatively from about 1.3 to about 3, and alternatively from about L3 to about 2.7 saccharide units. Any reducing saccharide containing 5 or 6 carbon atoms can be used, e.g., glucose, galactose and galactosyl moieties can be substituted for the glucosyl moieties. (Optionally the hydrophobic group is attached at the 2-,3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units. Optionally there can be a polyalkyleneoxide chain joining the hydrophobic moiety and the polysaccharide moiety. The alkyl group preferably contains up to about 3 hydroxy groups and/or the polyalkyleneoxide chain can contain up to about 10, preferably less than 5, alkylene moieties. Suitable alkyl polysaccharides are octyl, nonyldecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses and/or galactoses.

9. Polyethylene glycol (PEG) glyceryl fatty esters, as depicted by the formula RC(O)OCH2 CH(OH)CH2 (OCH2 CH2)n OH wherein n is from about 5 to about 200, preferably from about 20 to about 100, more preferably from about 30 to about 85, and RC(O)— is an ester wherein R comprises an aliphatic radical having from about 7 to 19 carbon atoms, preferably from about 9 to 17 carbon atoms, more preferably from about 11 to 17 carbon atoms, most preferably from about 11 to 14 carbon atoms. The combinations of n may be from about 20 to about 100, with C12-C18, alternatively C12-C15 fatty esters, for minimized adverse effect on foaming.

The nonionic emulsifier may be a silicone emulsifier. A wide variety of silicone emulsifiers may be useful herein. These silicone emulsifiers are typically organically modified siloxanes, also known to those skilled in the art as silicone surfactants. Useful silicone emulsifiers can include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols can include materials having various cationic, anionic, amphoteric, and zwitterionic pendant moieties.

The nonionic emulsifier may have a hydrocarbon chain length of from about 16 to about 20 carbon atoms and from about 20 to about 25 moles of ethoxylate.

The nonionic emulsifier may have a hydrocarbon chain length of from about 19 to about 11, alternatively from about 9 to about 11 carbon atoms, and from about 2 to about 4 moles of ethoxylate.

The nonionic emulsifier may comprise a combination of (a) a nonionic emulsifier having a hydrocarbon chain that is branched, has a length of from about 11 to about 15 carbon atoms, and has from about 5 to about 9 moles of ethoxylate; and (b) a nonionic emulsifier having a hydrocarbon chain that has a length of from about 11 to about 13 carbon atoms and has from about 9 to about 12 moles of ethoxylate.

The nanoemulsions used in this invention may be prepared by two different methods: (1) mechanical, and (2) emulsion polymerization.

The first method of preparing the nanoemulsion is the mechanical method in which the nanoemulsion is prepared via the following steps: (1) a primary surfactant is dissolved in water, (2) a silicone is added, and a two-phase mixture is formed, (3) with simple mixing, a co-surfactant is slowly added to the two-phase mixture, until a clear isotropic microemulsion of a siloxane-in-water is formed.

The second method of preparing the nanoemulsion is by emulsion polymerization. Emulsion polymerization methods for making nanoemulsions of polymers involve starting with polymer precursors, i.e., monomers, or reactive oligomers, which are immiscible in water; a surfactant to stabilize polymer precursor droplets in water; and a water soluble polymerization catalyst. Typically, the catalyst is a strong mineral acid such as hydrochloric acid, or a strong alkaline catalyst such as sodium hydroxide. These components are added to water, the mixture is stirred, and polymerization is allowed to advance until the reaction is complete, or the desired degree of polymerization (DP) is reached, and an emulsion of the polymer is formed.

E. Perfume

The hair care composition may comprise from about 0.75% to about 7%, alternatively from about 1% to about 6%, alternatively from about 1.5% to about 5% perfume, alternatively from about 1.25% to about 4% perfume, and alternatively from about 2% to about 3.5% by weight of the hair care composition.

The pressurized hair care composition may comprise from about 0.75% to about 7%, alternatively from about 1% to about 6%, alternatively from about 1.5% to about 5% perfume, alternatively from about 1.25% to about 4% perfume, and alternatively from about 2% to about 3.5% by weight of the pressurized hair care composition.

The hair care composition can have a silicone to perfume ratio of from about 98:2 to about 50:50, alternatively from about 90:10 to about 55:45, alternatively from about about 85:15 to about 60:40, and alternatively from about 80:20 to about 65:35. The hair care composition can have a perfume to silicone ratio of from about 2:98 to about 1:1, alternatively from about 10:90 to about 45:55, alternatively from about 15:85 to about 40:60, and alternatively from about 20:80 to about 35:65.

The pressurized hair care composition can have a silicone to perfume ratio of from about 98:2 to about 50:50, alternatively from about 90:10 to 55:45, alternatively from about 85:15 to 60:40, and alternatively from about 80:20 to about 65:35. The pressurized hair care composition can have a perfume to silicone ratio of from about 2:98 to about 1, alternatively from about 10:90 to about 45:55, alternatively from about 15:85 to about 40:60, and alternatively from about 20:80 to about 35:65.

Examples of suitable perfumes may be provided in the CTFA (Cosmetic, Toiletry and Fragrance Association) 1992 International Buyers Guide, published by CFTA Publications and OPD 1993 Chemicals Buyers Directory 80th Annual Edition, published by Schnell Publishing Co. A plurality of perfume components may be present in the hair care composition and the pressurized hair care composition.

F. High Melting Point Fatty Compounds

The hair care composition can comprise less than 8%, alternatively less than 6% high melting point fatty compounds, alternatively less than 5% high melting point fatty compounds, alternatively less than 4% high melting point fatty compounds, alternatively less than 3% high melting point fatty compound, alternatively may be substantially free of high melting point fatty compounds, and alternatively may comprise 0% high melting point fatty compounds, by weight of the hair care composition. The hair care composition can comprise from about 0% to about 6% fatty alcohols, alternatively from about 0.5% to about 5%, alternatively from about 1% to about 4%, and alternatively from about 1.5% to about 3.0%, by weight of the hair care composition. The hair care composition can have a silicone to high melting point fatty compounds weight ratio of from about 100:0 to about 45:55, alternatively from about 100:0 to about 50:50, and alternatively from about 100:0 to about 60:40. The hair care composition can have a silicone to high melting point fatty compounds weight ratio of from about 100:0 to about 70:30. The hair care composition can have a high melting point fatty compounds to silicone weight ratio of from about 0 to about 55:45, alternatively from about 0 to about 1, and alternatively from about 0 to about 40:60. The hair care composition can have a high melting point fatty compounds to silicone weight ratio of from about 0 to about 30:70.

The pressurized hair care composition can comprise less than 7.5% high melting point fatty compounds, alternatively less than 5% high melting point fatty compounds, alternatively less than 4% high melting point fatty compounds, alternatively less than 3% high melting point fatty compound, alternatively may be substantially free of high melting point fatty compounds, and alternatively may comprise 0% high melting point fatty compounds, by weight of the pressurized hair care composition. The pressurized hair care composition can comprise from about 0% to about 6%, alternatively from about 0.5% to about 5%, alternatively from about 1% to about 4%, and alternatively from about 1.5% to about 3.0% fatty alcohols, by weight of the pressurized hair care composition. The pressurized hair care composition can have a silicone to high melting point fatty compounds weight ratio of from about 100:0 to about 45:55, alternatively from about 100:0 to about 50:50, and alternatively from about 100:0 to about 60:40. The pressurized hair care composition can have a silicone to high melting point fatty compounds weight ratio of from about 100:0 to about 70:30. The pressurized hair care composition can have a high melting point fatty compounds to silicone weight ratio of from about 0 to about 55:45, alternatively from about 0 to about 1:1, and alternatively from about 0 to about 40:60. The pressurized hair care composition can have a high melting point fatty compounds to silicone weight ratio of from about 0 to about 30:70.

The hair care composition and/or the pressurized hair care composition can have a silicone to high melting point fatty compounds weight ratio of from about 100:0 to about 40:60 when the hair care composition and/or the pressurized hair care composition comprises from about 3 wt. % to about 8 wt. % silicone. The hair care composition and/or the pressurized hair care composition can have a high melting point fatty compounds to silicone weight ratio of from about 0 to about 60:40 when the hair care composition and/or the pressurized hair care composition comprises from about 3 wt. % to about 8 wt. % silicone.

The hair care composition and/or the pressurized hair care composition can have a silicone to high melting point fatty compounds weight ratio of from about 100:0 to about 50:50 when the hair care composition and/or the pressurized hair care composition comprises from about 3 wt. % to about 12 wt. % silicone. The hair care composition and/or the pressurized hair care composition can have a high melting point fatty compounds to silicone weight ratio of from about 0 to about 1 when the hair care composition and/or the pressurized hair care composition comprises from about 3 wt. % to about 12 wt. % silicone.

The hair care composition and/or the pressurized hair care composition can have a silicone to high melting point fatty compounds weight ratio of from about 100:0 to about 60:40 when the hair care composition and/or the pressurized hair care composition comprises from about 3 wt. % to about 18 wt. % silicone. The hair care composition and/or the pressurized hair care composition can have a high melting point fatty compounds to silicone weight ratio of from about 0 to about 40:60 when the hair care composition and/or the pressurized hair care composition comprises from about 3 wt. % to about 18 wt. % silicone.

The high melting point fatty compounds have a melting point of about 25° C. or higher, and can be selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The fatty alcohols described herein are those having from about 14 to about 30 carbon atoms, preferably from about 16 to about 22 carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through steareth-10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e., a mixture of fatty alcohols containing predominantly cetyl and stearyl alcohol, wherein the numeric designation indicates the number of ethylene glycol moieties present; C16-C30 alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

The fatty compound may be a single high melting point compound of high purity. Single compounds of pure fatty alcohols selected may be selected from the group consisting of pure cetyl alcohol, stearyl alcohol, and behenyl alcohol. By “pure” herein, what is meant is that the compound has a purity of at least about 90%, alternatively at least about 95%.

Commercially available high melting point fatty compounds described herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago, Ill. USA), HYSTRENE available from Witco Corp. (Dublin, Ohio USA), and DERMA available from Vevy (Genova, Italy).

G. Cationic Surfactants

The hair care composition described herein can comprise 0%, alternatively less than 10%, alternatively less than 7.5%, alternatively less than 5%, alternatively less than 2.5%, alternatively from about 0.25% to about 10%, alternatively from about 0.5% to about 7.5%, alternatively from about 1% to about 6%, alternatively from about 2% to about 5%, alternatively from about 3% to about 6%, and alternatively from about 1% to about 3% cationic surfactants, by weight of the hair care composition.

The pressurized hair care composition described herein can comprise 0%, alternatively less than 9%, alternatively less than 7%, alternatively less than 5%, alternatively less than 2.5%, alternatively from about 0.25% to about 9%, alternatively from about 0.5% to about 7%, alternatively from about 1% to about 6%, alternatively from about 2% to about 5%, alternatively from about 3% to about 6%, and alternatively from about 1% to about 3% cationic surfactants, by weight of the pressurized hair care composition.

The cationic surfactant can be selected from the group consisting of mono-long alkyl quaternized ammonium salts, di-long alkyl quaternized ammonium salts, mono-long alkyl amidoamine salts, and mixtures thereof.

(i) Mono-Long Alkyl Quaternized Ammonium Salts

The cationic surfactant can be a mono-long alkyl quaternized ammonium salt having the formula (VII) [from WO2013148778]:

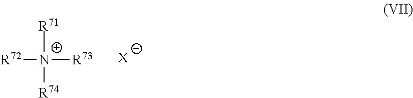

wherein one of R⁷¹, R⁷² R⁷³ a n R⁷⁴ selected from an aliphatic group of from about 14 to about 30 carbon atoms or an aromatic, alkoxy, polyoxyalkylene, alkylamido, hydroxyalkyl, aryl or alkylaryl group having up to about 30 carbon atoms; the remainder of R⁷¹, R⁷² R⁷³ and R⁷⁴ are independently selected from an aliphatic group of from about 1 to about 8 carbon atoms or an aromatic, alkoxy, polyoxyalkylene, alkylamido, hydroxyalkyl, aryl or alkylaryl group having up to about 8 carbon atoms; and X is a salt-forming anion such as those selected from halogen, (e.g., chloride, bromide), acetate, citrate, lactate, glycolate, phosphate, nitrate, sulfonate, sulfate, alkylsulfate, glutamate, and alkyl sulfonate radicals. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, one of R⁷¹, R⁷² R⁷³ and R⁷⁴ is selected from an alkyl group of from about 14 to about 30 carbon atoms, more preferably from about 16 to about 22 carbon atoms, still more preferably from about 16 to about 18 carbon atoms; the remainder of R⁷¹, R⁷², R⁷³, and R⁷⁴ are independently selected from the group consisting of CH₃, C₂H₅, C₂H₄OH, CH₂C₅H₅, and mixtures thereof; and (X) is selected from the group consisting of Cl, Br, CH₃OSO₃, and mixtures thereof. Mono-long alkyl quatemized ammonium salts can provide improved slippery and slick feel on wet hair.

Nonlimiting examples of such mono-long alkyl quatemized ammonium salt cationic surfactants include: behenyl trimethyl ammonium chloride available, for example, with tradename Genamine KDMP from Clariant, with tradename INCROQUAT TMC-80 from Croda and ECONOL TM22 from Sanyo Kasei; stearyl trimethyl ammonium chloride available, for example, with tradename CA-2450 from Nikko Chemicals; cetyl trimethyl ammonium chloride available, for example, with tradename CA-2350 from Nikko Chemicals; behenyltrimethylammonium methyl sulfate, available from FeiXiang; hydrogenated tallow alkyl trimethyl ammonium chloride; stearyl dimethyl benzyl ammonium chloride; and stearoyl amidopropyl dimethyl benzyl ammonium chloride.

(ii) Mono-Long Alkyl Amidoamine Salts

Mono-long alkyl amines can also be suitable as cationic surfactants. Primary, secondary, and tertiary fatty amines can be useful. The cationic surfactants can be tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Additional cationic surfactant amines are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein can be partially neutralized with any of the acids at a molar ratio of the amine to the acid of from about 1:0.3 to about 1:2, alternatively from about 1:0.4 to about 1:1.

(iii) Di-Long Alkyl Quaternized Ammonium Salts

The cationic surfactants described herein can be di-long alkyl quaternized ammonium salts. Di-long alkyl quaternized ammonium salts can be combined with a mono-long alkyl quaternized ammonium salt or mono-long alkyl amidoamine salt. Such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts can be used at a level such that the wt % of the dialkyl quaternized ammonium salt in the cationic surfactant system is in the range of from about 10% to about 50%, alternatively from about 30% to about 45%.

Di-alkyl cationic surfactants useful herein can be those having two long alkyl chains of from 12 to 30 carbon atoms, alternatively from 16 to 24 carbon atoms, alternatively from 16 to 22 carbon atoms, including, for example, di-long alkyl quaternized ammonium salts. Such di-alkyl quaternized ammonium salts useful herein can be those having the formula (VIII):

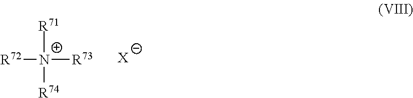

wherein two of R⁷¹, R⁷², R⁷³ and R⁷⁴ are selected from an aliphatic group of from 12 to 30 carbon atoms, preferably from 16 to 24 carbon atoms, more preferably from 16 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Two of R⁷¹, R⁷², R⁷³ and R⁷⁴ can be selected from an alkyl group of from 12 to 30 carbon atoms, alternatively from 16 to 24 carbon atoms, alternatively from 18 to 22 carbon atoms; and the remainder of R⁷¹, R⁷², R⁷³ and R⁷⁴ can be independently selected from CH₃, C₂H₅, C₂H₄OH, CH₂C₆H₅, and mixtures thereof.

Additional di-alkyl cationic surfactants can include dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

H. Water Miscible Solvents

The hair care composition described herein can comprise from about 0.1% to about 15%, alternatively from about 0.2% to about 10%, and alternatively from about 0.3% to about 5% of a water miscible solvent, by weight of the hair care composition. Alternatively, the hair care composition described herein can comprise from about 0.5% to about 10%, alternatively from about 0.75% to about 7.5%, alternatively from about 1% to about 5%, and alternatively from about 1.25% to about 3% of a water miscible solvent, by weight of the hair care composition.

The pressurized hair care composition described herein can comprise from about 0.1% to about 14%, alternatively from about 0.2% to about 9%, and alternatively from about 0.3% to about 5% of a water miscible solvent, by weight of the pressurized hair care composition. Alternatively, the pressurized hair care composition described herein can comprise from about 0.5% to about 9%, alternatively from about 0.75% to about 7%, alternatively from about 1% to about 5%, and alternatively from about 1.25% to about 3% of a water miscible solvent, by weight of the pressurized hair care composition.

Non-limiting examples of suitable water miscible solvents include polyols, copolyols, polycarboxylic acids, polyesters and alcohols.

Additional examples of useful polyols include, but are not limited to, glycerin, diglycerin, propylene glycol, ethylene glycol, butylene glycol, pentylene glycol, 1,3-butylene glycol, cyclohexane dimethanol, hexane diol, polyethylene glycol (200-600), sugar alcohols such as sorbitol, manitol, lactitol and other mono- and polyhydric low molecular weight alcohols (e.g., C₂-C₈ alcohols); mono di- and oligo-saccharides such as fructose, glucose, sucrose, maltose, lactose, and high fructose corn syrup solids and ascorbic acid.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Examples of suitable alcohols include, but are not limited to ethanol, n-propanol, isopropanol, n-butanol, sec-butanol, tert-butanol, n-hexanol and cyclohexanol.

Other suitable water miscible solvents include, but are not limited to, alkyl and allyl phthalates; napthalates; lactates (e.g., sodium, ammonium and potassium salts); sorbeth-30; urea; lactic acid; sodium pyrrolidone carboxylic acid (PCA); sodium hyraluronate or hyaluronic acid; soluble collagen; modified protein; monosodium L-glutamate; alpha & beta hydroxyl acids such as glycolic acid, lactic acid, citric acid, maleic acid and salicylic acid; glyceryl polymethacrylate; polymeric plasticizers such as polyquaterniums; proteins and amino acids such as glutamic acid, aspartic acid, and lysine; hydrogen starch hydrolysates; other low molecular weight esters (e.g., esters of C₂-C₁₀ alcohols and acids); and any other water soluble plasticizer known to one skilled in the art of the foods and plastics industries; and mixtures thereof.

The water miscible solvents may be selected from the group consisting of glycerin, propylene glycol, dipropylene glycol, and mixtures thereof. EP 0283165 B1 discloses other suitable water miscible solvents, including glycerol derivatives such as propoxylated glycerol. The water miscible solvent may be selected from glycerin.

I. Viscosity Modifiers

The hair care composition described herein can comprise from about 0.1% to about 2%, alternatively from about 0.1% to about 1%, and alternatively from about 0.1% to about 0.5% of a viscosity modifier, by weight of the hair care composition.

The pressurized hair care composition described herein can comprise from about 0.1% to about 2%, alternatively from about 0.1% to about 1%, and alternatively from about 0.1% to about 0.5% of a viscosity modifier, by weight of the pressurized hair care composition.

Non-limiting examples of suitable viscosity modifiers include water soluble polymers and cationic water soluble polymers.

Examples of water soluble polymers include, but are not limited to (1) vegetable based polymers such as gum Arabic, tragacanth gum, galactan, guar gum, carob gum, karaya gum, carrageenan, pectin, agar, quince seed, algal colloid, starch (rice, corn, potato, or wheat), and glycyrrhizinic acid; (2) microorganism-based polymers such as xanthan gum, dextran, succinoglucan, and pullulan; and (3) animal-based polymers such as collagen, casein, albumin, and gelatin. Examples of semi-synthetic water-soluble polymers include (1) starch-based polymers such as carboxymethyl starch and methylhydroxypropyl starch; (2) cellulose-based polymers such as methylcellulose, nitrocellulose, ethylcellulose, methylhydroxypropylcellulose, hydroxyethylcellulose, sodium cellulose sulfate, hydroxypropylcellulose, sodium carboxymethylcellulose (CMC), crystalline cellulose, and cellulose powder; and (3) alginate-based polymers such as sodium alginate and propylene glycol alginate. Examples of synthetic water-soluble polymers include (1) vinyl-based polymers such as polyvinyl alcohol, polyvinyl methyl ether-based polymer, polyvinylpyrrolidone, and carboxyvinyl polymer (CARBOPOL 940, CARBOPOL 941; (2) polyoxyethylene-based polymers such as polyethylene glycol 20,000, polyethylene glycol 6,000, and polyethylene glycol 4,000; (3) copolymer-based polymers such as a copolymer of polyoxyethylene and polyoxypropylene, and PEG/PPG methyl ether; (4) acryl-based polymers such as poly(sodium acrylate), poly(ethyl acrylate), polyacrylamide, polyethylene imines, and cationic polymers. The water-swellable clay minerals are nonionic water-soluble polymers and correspond to one type of colloid-containing aluminum silicate having a triple layer structure. More particular, as examples thereof, mention may be made of bentonite, montmorillonite, beidellite, nontronite, saponite, hectorite, aluminum magnesium silicate, and silicic anhydride.

Examples of cationic water soluble polymers include, but are not limited to (1) quaternary nitrogen-modified polysaccharides such as cation-modified cellulose, cation-modified hydroxyethylcellulose, cation-modified guar gum, cation-modified locust bean gum, and cation-modified starch; (2) dimethyldiallylammonium chloride derivatives such as a copolymer of dimethyldiallylammonium chloride and acrylamide, and poly(dimethylmethylene piperidinium chloride); (3) vinylpyrrolidone derivatives such as a salt of a copolymer of vinylpyrrolidone and dimethylaminoethyl methacrylic acid, a copolymer of vinylpyrrolidone and methacrylamide propyltrimethylammonium chloride, and a copolymer of vinylpyrrolidone and methylvinylimidazolium chloride; and (4) methacrylic acid derivatives such as a copolymer of methacryloylethyldimethylbetaine, methacryloylethyl trimethylammonium chloride and 2-hydroxyethyl methacrylate, a copolymer of methacryloylethyldimethylbetaine, and methacryloylethyl trimethylammonium chloride and methoxy polyethylene glycol methacrylate.

J. Viscosity

The hair care composition described herein can have a liquid phase viscosity of from about 1 centipoise to about 2,500 centipoise, alternatively from about 5 centipoise to about 2,000 centipoise, alternatively from about 10 centipoise to about 1,500 centipoise, and alternatively from about 15 centipoise to about 1,000 centipoise. Alternatively, the hair care composition described herein can have a liquid phase viscosity of from about 1 centipoise to about 15,000 centipoise, alternatively from about 1 centipoise to about 8,000 centipoise, alternatively from about 5 centipoise to about 5,000 centipoise, alternatively from about 10 centipoise to about 2,500 centipoise, alternatively from about 15 centipoise to about 1,500 centipoise, and alternatively from about 20 centipoise to about 1,000 centipoise. Alternatively, the hair care composition described herein may have a liquid phase viscosity of from about 200 centipoise to about 15,000 centipoise, alternatively from about 300 centipoise to about 12,000 centipoise, alternatively from about 400 centipoise to about 8,000 centipoise, alternatively from about 500 centipoise to about 5,000 centipoise, and alternatively from about 600 centipoise to about 2,500 centipoise, and alternatively from about 700 centipoise to about 2,000 centipoise.

The liquid phase viscosity values of the hair care composition described herein can be measured employing any suitable rheometer or viscometer at 25.0° C. and at a shear rate of about 2 reciprocal seconds. The liquid phase viscosity is measured prior to the addition of the propellant.

For example, the liquid phase viscosity values reported in the data herein were measured using a Cone/Plate Controlled Stress Brookfield Rheometer R/S Plus, by Brookfield Engineering Laboratories, Stoughton, Mass. The cone used (Spindle C-75-1) has a diameter of 75 mm and 1° angle. The liquid phase viscosity was determined using a steady state flow experiment at constant shear rate of 2 s⁻¹ and at temperature of 25.0° C. The sample size was 2.5 ml and the total measurement reading time was 3 minutes.

K. Optional Ingredients

The hair care composition described herein and the pressurized hair care composition described herein can optionally comprise one or more additional components known for use in hair care or personal care products, provided that the additional components are physically and chemically compatible with the essential components described herein, or do not otherwise unduly impair product stability, aesthetics or performance. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the conditioning composition.

Emulsifiers suitable as an optional ingredient herein include mono- and di-glycerides, fatty alcohols, polyglycerol esters, propylene glycol esters, sorbitan esters and other emulsifiers known or otherwise commonly used to stabilized air interfaces, as for example those used during preparation of aerated foodstuffs such as cakes and other baked goods and confectionary products, or the stabilization of cosmetics such as hair mousses.

Further non-limiting examples of such optional ingredients include preservatives, perfumes or fragrances, cationic polymers, viscosity modifiers, coloring agents or dyes, conditioning agents, hair bleaching agents, thickeners, moisturizers, foam boosters, additional surfactants or nonionic cosurfactants, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, non-volatile solvents or diluents (water-soluble and water-insoluble), co-solvents or other additional solvents, and similar other materials.

L. Aerosol Dispenser

The aerosol dispenser may comprise a reservoir for holding the hair care composition and/or the pressurized hair care composition. The reservoir may be made out of any suitable material selected from the group consisting of plastic, metal, alloy, laminate, and combinations thereof. The reservoir may be for one-time use. The reservoir may be removable from the aerosol dispenser. Alternatively, the reservoir may be integrated with the aerosol dispenser. Alternatively, there may be two or more reservoirs.

The reservoir may be comprised of a material selected from the group consisting of rigid materials, flexible materials, and combinations thereof. The reservoir may be comprised of a rigid material if it does not collapse under external atmospheric pressure when it is subject to an interior partial vacuum.

The aerosol dispenser may comprise a dip-tube to enable upright dispensing.

The aerosol dispenser may be of the bag on valve type wherein the container comprises an inner bag and an outer container, which encloses the inner bag, while the inner bag has a valve mechanism attached which is movable between an open position and a closed position. The outer container may be formed from metal or plastic or the like, and any of the propellants described herein can be filled in a space between the outer container and the inner bag. The inner bag may be flexible, and can be made from a single material or from a composite material including plastic, which may comprise at least a polymeric layer and a layer which acts as a gas barrier, e.g., made from metal, such as Aluminum. The inner material of the bag may be inert to the contents of the composition, and the inner material may also be impenetrable by the contents of the composition in the bag. The inner bag may comprise a layer of a material which is essentially impermeable to the propellant inside of the bag. The inner bag may comprise a layer of a material which is essentially impermeable to the propellant outside of the bag which generally is not intended to be mixed with the composition in the inner bag during storage. The propellant may be known as a foaming agent.

The foam has a dosage weight of from about 1 g to about 5 g when dispensed from the aerosol dispenser. The foam has a dosage weight of from about 1 g to about 7 g when dispensed from the aerosol dispenser, alternatively from about 2 g to about 6 g, alternatively from about 2.5 g to about 5 g, and alternatively from about 3 g to about 4.5 g. The dosage can be obtained via a single squeeze or actuation of the aerosol dispenser, but may be accomplished via two squeezes or actuations of the aerosol dispenser.

The pressure inside the aerosol dispenser can be from about 10 psig to about 100 psig, alternatively from about 20 psig to about 90 psig, alternatively from about 30 psig to about 80 psig, alternatively from about 40 psig to about 70 psig, alternatively from about 45 psig to about 65 psig, alternatively from about 30 psig to about 100 psig, alternatively from about 40 psig to about 90 psig, alternatively from about 45 psig to about 80 psig, alternatively from about 50 psig to about 70 psig, alternatively from about 20 psig to about 80 psig, alternatively from about 30 psig to about 60 psig, alternatively from about 40 psig to about 60 psig, alternatively from about 10 psig to about 50 psig, alternatively from about 45 psig to about 60 psig, alternatively from about 30 psig to about 50 psig, alternatively from about 20 psig to about 40 psig, and alternatively from about 50 psig to about 60 psig.

H. Propellant

A propellant can be added to the hair care composition described herein at a hair care composition to propellant weight ratio of from about 85:15 to about 98:2; alternatively from about 90:10 to about 97:3; and alternatively from about 92:8 to about 96:4 to create a pressurized hair care composition.

The pressurized hair care composition can comprise from about 1% to about 12% propellant, alternatively from about 2% to about 10% propellant, alternatively from about 3% to about 8% propellant, alternatively from about 4% to about 6% propellant, from about 1% to about 6% propellant, alternatively from about 2% to about 5% propellant, and alternatively from about 3% to about 4% propellant, by weight of the pressurized hair care composition.

The pressurized hair care composition can be dispensed as a foam wherein the foam has a density of from about 0.025 g/cm³ to about 0.30 g/cm³, alternatively from about 0.035 g/cm³ to about 0.20 g/cm³, alternatively from about 0.045 g/cm³ to about 0.15 g/cm³, and alternatively from about 0.055 g/cm³ to about 0.12 g/cm³. The pressurized hair care composition can be dispensed as a foam wherein the foam can have a density of from about 0.025 g/cm³ to about 0.40 g/cm³, alternatively from about 0.035 g/cm³ to about 0.30 g/cm³, alternatively from about 0.025 g/cm³ to about 0.20 g/cm³, alternatively from about 0.045 g/cm³ to about 0.20 g/cm³, alternatively from about 0.045 g/cm³ to about 0.15 g/cm³, alternatively from about 0.055 g/cm³ to about 0.15 g/cm³, and alternatively from about 0.075 g/cm³ to about 0.15 g/cm³.

The propellant may comprise one or more volatile materials, which in a gaseous state, may carry the other components of the pressurized hair care composition in particulate or droplet form. The propellant may have a boiling point within the range of from about −45° C. to about 5° C. The propellant may be liquefied when packaged in convention aerosol containers under pressure. The rapid boiling of the propellant upon leaving the aerosol dispenser may aid in the atomization of the other components of the pressurized hair care composition.

The propellant which may be employed in the hair care compositions described herein can include the chemically-inert hydrocarbons such as propane, n-butane, isobutane, n-pentane, isopentane, and mixtures thereof; chlorofluorocarbons (CFCs) such as 20 dichlorodifluoromethane, 1,1-dichloro-1,1,2,2-tetrafluoroethane, 1-chloro-1,1-difluoro-2,2-trifluoroethane, 1-chloro-1,1-difluoroethylene, monochlorodifluoromethane and mixtures thereof; hydrofluorocarbons (HFCs) such as 1,1-difluoroethane, 1,3,3,3-tetrafluoropropene, 2,3,3,3-tetrafluoropropene and mixtures thereof; hydrofluoroolefins (HFOs) such as 2,3,3,3-tetrafluoropropene (HFO-1234yf), 1,3,3,3-tetrafluoropropene (HFO-1234ze), and mixtures 25 thereof; alkyl ethers such as dimethyl ether, methyl ethyl ether, and mixtures thereof; compressed gases such as carbon dioxide, nitrous oxide, nitrogen, compressed air, and mixtures thereof; and mixtures of one ar more hydrocarbons, chlorofluorocarbons, hydrofluorocarbons, hydrofluoroolefins, alkyl ethers, and compressed gases. The propellant can be 1,3,3,3-hydrofluoropropene.

The propellant can also comprise a blend of hydrocarbons such as isobutane, 30 propane, and butane including, but not limited to, hydrocarbon blend A-46 (15.2% propane, 84.8% isobutane), hydrocarbon blend NP-46 (25.9% propane, 74.1% n-butane hydrocarbon blend NIP-46 (21.9% propane, 31.3% isobutane, 46.8% n-butane), and other non-limiting hydrocarbon blends designated as A-31, NP-31, NIP-31, A-70, NP-70, NIP-70, A-85, NP-85, A-108. The propellant may include chlorofluorocarbons (CFCs) including, but not limited to 1,1-dichloroethane (WC-152a). The propellant may include hydrofluorocarbons (HFCs) including, but not limited to, 1,3,3,3-tetrafluoropropene (HFC-134a). The propellant may include hydrofluoroolefins (HFOs) including, but not limited to, 2,3,3,3-5 tetrafluoropropene (HFO-1234yf), and 1,3,3,3-tetrafluoropropene (HFO-1234ze). The propellant can include compressed gases including, but not limited to, carbon dioxide and nitrous oxide.

I. Water

The hair care composition described herein may comprise from about from about 60% to about 90% water, alternatively from about 65% to about 87.5%, alternatively from about 67.5% to about 85%, alternatively from about 70% to about 82.5%, and alternatively from about 72.5% to about 80%, by weight of the hair care composition.

The pressurized hair care composition described herein may comprise from about from about 55% to about 87% water, alternatively from about 62% to about 85%, alternatively from about 65% to about 83%, alternatively from about 68% to about 80%, and alternatively from about 70% to about 78%, by weight of the pressurized hair care composition.

J. Method of Conditioning the Hair

The method of conditioning the hair described herein comprises (1) providing a hair care composition as described herein; (2) adding a propellant to the hair care composition to create a pressurized hair care composition; (2) dispensing the pressurized hair care composition from an aerosol dispenser as a dosage of foam; (3) applying the foam to the hair; and (4) rinsing the foam from the hair.

### DATA & EXAMPLES

The following data and examples illustrate the hair care composition and/or pressurized hair care composition and/or method of conditioning the hair described herein. The exemplified compositions can be prepared by conventional formulation and mixing techniques. It will be appreciated that other modifications within the skill of those in the conditioner formulation art can be undertaken. All parts, percentages, and ratios herein are by weight unless otherwise specified. Some components may come from suppliers as dilute solutions. The amount stated can reflect the weight percent of the active material, unless otherwise specified.

### Data:

To prepare human hair for application of the hair care compositions from Table 2, three “Clarifying” shampoos were employed that were void of high melting point fatty compounds and conditioning agents. One was a Pantene clarifying shampoo, and the other two from Table 1 were concentrated foam shampoos. The concentrated foam shampoos were prepared by mixing together water and surfactants along with any solids that needed to be melted at an elevated temperature, e.g. about 75° C. The ingredients were mixed thoroughly at the elevated temperature and then cooled to ambient temperature. Any additional ingredients, including electrolytes, preservatives and fragrances, were added to the cooled product.

TABLE 1
Aerosol Foam Cleansing Shampoo Compositions

| | Foam | Foam |
|---|---|---|
| Raw Material | Shampoo 1 | Shampoo 2 |
| Sodium Undecyl Sulfate (C11 70% active)¹ | 23.6 | |
| Lauramidopropyl Betaine | 5.8 | |
| (LAPB 35% active)² | | |
| Para Hydroxy Phenyl Butanone³ | 3.9 | |
| Alkyl polyglucoside⁴ | | 25.2 |
| Cocamidopropyl betaine | | 3.9 |
| Polyvinyl alcohol⁵ | 1.9 | 1.9 |
| Perfume | 2.3 | 2.3 |
| Citric Acid | 0.3 | 0.3 |
| Preservative (Kathon) | 0.03 | 0.03 |
| Propellant (Aeron-46) | 3.1 | 3.1 |
| Water | q.s. | q.s. |
| Weight % of high melting point | 0% | 0% |
| fatty compounds | | |

¹Sodium Undecyl Sulfate (C11, Isachem 123S) at 70% active, supplier: P&G
²LAPB (Mackam DAB), at 35% active level, supplier: Rhodia
³Raspberry Ketone, supplier: Spectrum
⁴EcoSense 919 available from Dow Chemical.
⁵PVA-403 available from Kuraray

The hair care compositions from Table 2 were prepared by weighing distilled water and the aminosilicone emulsions into a stainless steel beaker. The beaker was placed in a water bath on a hot plate while mixing with overhead mixer at 100 to 150 rpm. If fatty alcohols present in the formula were added, and the mixture was heated to 70-75° C. Cetyltrimethylammonium choloride was then added and the mixing speed was increased to 250-350 rpm due to viscosity increase. When the materials were all heated thoroughly and were homogenous, the heating was stopped while the mixture continued to be stirred. The batch was cooled to 35° C. by removing the hot water from the water bath and replacing with cold water. The perfume and Kathon were added with continued stirring for ˜10 minutes.

Propellant Aeron-46 was added to each of the below hair care compositions at a weight ratio of 4 parts Aeron-46 to 96 parts of formula within an aerosol container to create a pressurized hair care composition.

Examples 1 through 6 from Table 2 are embodiments of the hair care composition described herein. Example 7 is a comparative example.

The ability to foam for examples 1 through 7 was assessed by shaking the aerosol container for 10 seconds and then seeing if 5 grams of foam could be dispensed into a weigh boat. The foam examples were qualitatively assessed by spreading the foam and assessing the ability to spread without foam collapse.

TABLE 2
Hair Care Compositions before Addition of Propellant

| Raw Material | Ex 1 | Ex 2 | Ex 3 | Ex 4 | Ex 5 | Ex 6 | Ex 7 |
|---|---|---|---|---|---|---|---|
| Aminosilicone (Si)¹ | 12 | 12 | 12 | 12 | 12 | 12 | 12 |
| Alcohol ethoxylates (NE)¹ | 7.5 | 7.5 | 7.5 | 7.5 | 7.5 | 7.5 | 7.5 |
| Glycerine (WMS)¹ | 1.8 | 1.8 | 1.8 | 1.8 | 1.8 | 1.8 | 1.8 |
| Perfume (Perf) | 2.4 | 2.4 | 3.0 | 3.0 | 3.0 | 3.0 | 3.0 |
| Cetyltrimethylammonium | | 2.5 | 2.5 | 2.5 | 2.5 | 2.5 | 2.5 |
| Chloride (CS) | | | | | | | |
| Cetyl Alcohol (HMPFC) | | | 1.5 | 3.0 | 4.0 | 6.0 | 9 |
| Stearyl Alcohol (HMPFC) | | | 1.5 | 3.0 | 4.0 | 6.0 | 9 |
| Preservative (Kathon) | 0.03 | 0.03 | 0.03 | 0.03 | 0.03 | 0.03 | 0.03 |
| Water | (q.s.) | (q.s.) | (q.s.) | (q.s.) | (q.s.) | (q.s.) | (q.s.) |
| Silicone Level (Si) | 12 | 12 | 12 | 12 | 12 | 12 | 12 |
| High Melting Point Fatty | 0 | 0 | 3.0 | 6.0 | 8.0 | 12.0 | 18.0 |
| Compound Level (HMPFC) | | | | | | | |
| Cationic Surfactant Level | 0 | 2.5 | 2.5 | 2.5 | 2.5 | 2.5 | 2.5 |
| (CS) | | | | | | | |
| Nonionic Emulsifiers (NE) | 7.5 | 7.5 | 7.5 | 7.5 | 7.5 | 7.5 | 7.5 |
| Perfume Level (Perf) | 2.4 | 2.4 | 3.0 | 3.0 | 3.0 | 3.0 | 3.0 |
| Water Miscible Solvent | 1.8 | 1.8 | 1.8 | 1.8 | 1.8 | 1.8 | 1.8 |
| (WMS) | | | | | | | |
| Weight ratio of silicone to | 100:0 | 100:0 | 80:20 | 67:33 | 60:40 | 50:50 | 40:60 |
| high melting point fatty | | | | | | | |
| compounds (Si:HMPFC) | | | | | | | |
| Weight ratio of high melting | 0:100 | 0:100 | 20:80 | 33:67 | 40:60 | 50:50 | 60:40 |
| point fatty compounds to | | | | | | | |
| silicone (HMPFC:Si) | | | | | | | |
| Weight ratio of silicone to | 83:17 | 83:17 | 80:20 | 80:20 | 80:20 | 80:20 | 80:20 |
| perfume (Si:Perf) | | | | | | | |
| Viscosity (cp) | <200 | <200 | 810 | 1,740 | 5,450 | 12,900 | 33,400 |
| Ability to dispense foam | Yes | Yes | Yes | Yes | Yes | Yes | No |
| after addition of propellant | | | | | | | |
| Foam quality after addition | Highly | Highly | Highly | Highly | Accept- | Accept- | Not |
| of propellant | Accept- | Accept- | Accept- | Accept- | able | able | Accept- |
| | able | able | able | able | | | able |

1 Silsoft 253 (20% active) nano-emulsion available from Momentive (10-20 nm), comprising secondary alcohol ethoxylate (5-10%), C12-16 synthetic alcohols-ethoxylated (1-5%), glycerine (1-5%), and tridecyl alcohol ethoxylate (1-5%)—computed average alcohol ethoxylates emulsifier level of 12.5%; computed average glycerine level of 3.0%.

Table 2 shows that as the ratio of high melting point fatty compounds to silicone increases, the ability to dispense foam and deliver acceptable foam quality decreases. This ratio also affects viscosity. Above a high melting point fatty compound to silicone ratio of about 50:50, the foam was not able to be dispensed (formula was too viscous). Therefore, Examples 1 through 4, which had a weight ratio of HMPFC:Si from about 0 to about 33:67, had highly acceptable foam quality, were progressed for performance testing as described in the data below.

In the following data, seven different shampoo+conditioner legs were tested. Examples 1 through 4 from Table 2 were pressurized by adding propellant Aeron-46 as described above as part of a regimen with Pantene Pro-V Clarifying Shampoo. Example 2 from Table 2 was pressurized by adding propellant Aeron-46 as described above and paired with Foam Shampoo 1 and Foam Shampoo 2 from Table 1. As a control, Pantene Pro-V Clarifying Shampoo was combined with Pantene Anti-Breakage Conditioner.

Pantene Anti-Breakage Conditioner has an aminosilicone content of 2.5% and a total high melting point fatty compounds (cetyl and stearyl alcohols) content of 5.20% for a weight ratio of silicone to high melting point fatty compounds of 32.5:67.5.

### Shampoo Plus Conditioner Treatment Method:

1. Six 4 gram, 8 inch General Population brown hair switches are wet with 100 degrees Fahrenheit water at a sink (bound on root-ends with glue/tape and hanging on metal holder) with a shower head fixture (flow rate is 1.5 gallons per minute) for 15 to 20 seconds.
2. Liquid shampoos are applied at 0.1 grams of product per gram of hair (e.g., Pantene Pro-V Clarifying Shampoo) via a syringe and milked/scrubbed for 30 seconds followed by a 30 seconds shower head rinse (with gentle manipulation at top of switch to ensure uniform rinsing). Concentrated liquid foam shampoos are applied at 0.05 grams of product per gram of hair with a spatula (foam is dispensed in weigh boat and applied weight recorded) and following the same application procedure.
3. Liquid conditioners are applied at a 0.1 grams of product per gram of hair (e.g., Pantene Moisture Renewal Conditioner etc.) via a syringe (weighed on weigh scale) evenly over the hair switch and milked/scrubbed for 30 seconds followed by a 30 seconds shower rinse (with gentle manipulation at top of switch to ensure uniform rinsing). Concentrated liquid foam conditioners are applied at 0.033 grams of product per gram of hair with a spatula (foam is dispensed in weigh boat and applied weight recorded) and following the same application procedure.
4. The hair is then dried in a heat box set at 60 C for ˜45 minutes or until mostly dry.
5. For multiple cycle testing, the above procedure is repeated for a set number of times. For instance, for a six cycle test, the above steps 1-4 are repeated six times.

### Silicone Deposition Purity Test Method:

Silicone Deposition Purity is determined by the ratio of silicone deposited per weight of hair to the total deposition of other ingredients per weight of hair. Silicone is determined by either extraction or digestion of the hair followed by an analysis with a quantitative elemental technique such as ICP for total silicon and converting to silicone based on the % of silicon in the silicone by weight. The total deposition may be determined by the sum of separate deposition measurements or by a Single Inclusive Measurement of total deposition. The separate deposition measurements may include but are not limited to: fatty alcohols, EGDS, quaternized agents and silicone. Typically these measurements involve extracting the hair then separating the ingredients of interest with chromatography and quantifying with an externally calibration based on test solution concentration. The Single Inclusive Measurement of total deposition is gravimetric. The hair is thoroughly extracted and the residue determined by weighing the dissolved residue in the extract after evaporating the solvent. This residue contains both deposited ingredients and naturally occurring extractable compounds from the hair (primarily lipids). The naturally occurring extractable compounds are quantified and subtracted from the total. These include: fatty acids, squalene, cholesterol, ceramides, wax esters, triglycerides and sterol esters. The method of quantitation is similar to the deposition measurements. Other supporting evidence of Deposition Purity may include spectroscopic or topography mapping of the hair surface.

Silicone Deposition (ppm) Test Method:

Hair samples treated with different products are submitted as balls of hair with an average sample size of 0.1 g. These hair samples are then digested using a single reaction chamber microwave digestion system (Milestone Inc., Shelton, Conn.) using a 6:1 HNO₃:H₂O₂ mixture and an aliquot of methyl isobutyl ketone (MIBK) in Teflon digestion vessels. A gentle digestion program with a ramp to 95° C. and a manual vent after cooling below 30° C. is used to facilitate retention of silicon. After dilution to volume, the samples are run against an inorganic silicon calibration curve produced on an Optima 8300 ICP-OES system (Perkin Elmer, Waltham, Mass.) run in the axial mode. The silicon values determined are converted to a concentration of silicone polymer-equivalents deposited on the hair sample using the theoretical silicon concentration of the polymer provided by the manufacturer. An untreated hair sample is analyzed to determine the background concentration of silicon to allow correction if needed. Another untreated hair sample is spiked with a known amount of polymer and analyzed to ensure recovery of the polymer and verify the analysis.

### Fatty Alcohol Deposition Test Method:

The weight of the fatty alcohol deposited onto the hair can be determined by extraction followed by quantitation via capillary gas chromatography. The resulting peaks are integrated and μg fatty alcohol/g of hair (ppm) is calculated using the internal standard mode.

Cationic Surfactant Deposition (ppm) Test Method:

The weight of the cationic surfactant deposited onto the hair can be determined by extraction followed by quantitation via ion exchange chromatography using a charged aerosol detector. The resulting peaks are integrated and μg cationic surfactant/g of hair (ppm) is calculated using the internal standard mode.

Total Deposition (ppm) Test Method:

Total deposition (ppm) is the summation of the fatty alcohol deposition (ppm), silicone deposition (ppm), and cationic surfactant deposition (ppm).

### General Population Hair

TABLE 3
Results of Deposition and Silicone Deposition Purity on General Population Hair after 6 Treatment Cycles

| | | | | Silicone | Fatty | | |
|---|---|---|---|---|---|---|---|
| | Fatty | Silicone | Cationic | to Fatty | Alcohol to | | |
| | Alcohol | Deposition | Surfactant | Alcohol | Silicone | Silicone | Total |
| | Deposition | (ppm) | Deposition | Deposition | Deposition | Deposition | Deposition |
| Regimen | (ppm) | [% RSD] | (ppm) | Weight Ratio | Weight Ratio | Purity | (ppm) |
| Clarifying | 1749 +/− 81 | 1557 +/− 844 | 26 +/− 2 | 47:53 | 53:47 | 47% | 3332 |
| Shampoo plus | | [54%] | | | | | |
| Pantene Anti- | | | | | | | |
| breakage | | | | | | | |
| Conditioner | | | | | | | |
| Clarifying | 3.0 +/− 8 | 1341 +/− 449 | 6 +/− 0.4 | 100:0 | 0 | 99% | 1350 |
| Shampoo plus Ex | | [33%] | | | | | |
| 1 Conditioner | | | | | | | |
| Clarifying | 14 +/− 4 | 1040 +/− 105 | 140 +/− 13 | 99:1 | 1:99 | 87% | 1194 |
| Shampoo plus Ex | | [10%] | | | | | |
| 2 Conditioner | | | | | | | |
| Clarifying | 488 +/− 23 | 1334 +/− 82 | 94 +/− 13 | 73:27 | 27:73 | 70% | 1916 |
| Shampoo plus Ex | | [6%] | | | | | |
| 3 Conditioner | | | | | | | |
| Clarifying | 668 +/− 36 | 1546 +/− 67 | 67 +/− 10 | 70:30 | 30:70 | 68% | 2281 |
| Shampoo plus Ex | | [4%] | | | | | |
| 4 Conditioner | | | | | | | |
| Foam Shampoo 1 | 8 +/− 3 | 934 +/− 60 | 126 +/− 9 | 100:0 | 0 | 87% | 1068 |
| plus Ex 2 | | [6%] | | | | | |
| Conditioner | | | | | | | |
| Foam Shampoo 2 | 1 +/− 5 | 996 +/− 58 | 158 +/− 12 | 100:0 | 0 | 86% | 1155 |
| plus Ex 2 | | [4%] | | | | | |
| Conditioner | | | | | | | |

In Table 3, the data demonstrates that the legs comprising the pressurized hair care compositions as described herein deposit efficacious levels of silicone onto the hair, but importantly at only ½ of the dosage (due to foam's lower product density) as the liquid control regimen (0.05 grams of product per gram of hair versus 0.1 grams of product per gram of hair) and with significantly less fatty alcohol co-deposits. Correspondingly, the measured fatty alcohol deposition to silicone deposition ratio was lower for the legs comprising the pressurized hair care compositions as described herein (from 0 to 30:70) versus the liquid control leg (53:47). The silicone deposition purity was generally higher and more consistent for the legs comprising the pressurized hair care compositions as described herein (68% to 99% purity) versus the liquid control leg (47% purity).

### Dyed Hair

TABLE 4
Results of Deposition and Silicone Deposition Purity on Dyed Hair after 6 Treatment Cycles

| | | | | Silicone- | Fatty | | | Dyed-to- |
|---|---|---|---|---|---|---|---|---|
| | Fatty | | Cationic | to-Fatty | Alcohol to | | | General |
| | Alcohol | Silicone | Surfactant | Alcohol | Silicone | Silicone | Total | Population |
| | Deposition | Deposition | Deposition | Deposition | Deposition | Deposition | Deposition | Hair Deposition |
| Regimen | (ppm) | (ppm) | (ppm) | Weight Ratio | Weight Ratio | Purity | (ppm) | Ratio (×100%) |
| Clarifying | 1532 +/− 84 | 1057 +/− 260 | 32 +/− 3 | 41:59 | 59:41 | 40% | 2621 | 68% |
| Shampoo plus | | [25%] | | | | | | |
| Pantene Anti- | | | | | | | | |
| breakage | | | | | | | | |
| Conditioner | | | | | | | | |
| Clarifying | 31 +/− 16 | 1464 +/− 78 | 11 +/− 0 | 98:2 | 2:98 | 97% | 1506 | 109% |
| Shampoo plus Ex | | [5%] | | | | | | |
| 1 Conditioner | | | | | | | | |
| Clarifying | 31 +/− 16 | 1385 +/− 149 | 149 +/− 26 | 98:2 | 2:98 | 88% | 1565 | 133% |
| Shampoo plus Ex | | [11%] | | | | | | |
| 2 Conditioner | | | | | | | | |
| Clarifying | 444 +/− 12 | 2040 +/− 147 | 115 +/− 25 | 82:18 | 18:82 | 78% | 2599 | 153% |
| Shampoo plus Ex | | [7%] | | | | | | |
| 3 Conditioner | | | | | | | | |
| Clarifying | 636 +/− 41 | 2225 +/− 108 | 84 +/− 13 | 78:22 | 22:78 | 76% | 2945 | 144% |
| Shampoo plus Ex | | [5%] | | | | | | |
| 4 Conditioner | | | | | | | | |
| Foam Shampoo 1 | 7 +/− 17 | 1484 +/− 47 | 133 +/− 15 | 100:0 | 0:100 | 91% | 1624 | 159% |
| plus Ex 2 | | [3%] | | | | | | |
| Conditioner | | | | | | | | |
| Foam Shampoo 2 | 9 +/− 10 | 1479 +/− 103 | 195 +/− 16 | 100:0 | 0:100 | 88% | 1683 | 148% |
| plus Ex 2 | | [7%] | | | | | | |
| Conditioner | | | | | | | | |

In Table 4, the data demonstrates that the legs comprising the pressurized hair care compositions as described herein deposit efficacious levels of silicone onto hair, but importantly at only ½ of the dosage (due to foam's lower product density) as the liquid control regimen (0.05 grams of product per gram of hair versus 0.1 grams of product per gram of hair) and with significantly less fatty alcohol co-deposits. Correspondingly, the measured fatty alcohol deposition to silicone ratio was lower for the legs comprising the pressurized hair care compositions as described herein (from 0 to 22:78) versus the liquid control leg (59:41). The silicone deposition purity was generally higher and more consistent for the legs comprising the pressurized hair care compositions as described herein (76% to 97% purity) versus the liquid control leg (40% purity). Moreover, the legs comprising the pressurized hair care compositions as described herein also generally deposit a greater amount of silicone onto the more polar dyed hair than on general population hair (dyed to general population hair deposition ratios of 109% to 159%) versus the liquid control leg which deposited less silicone on the more polar dyed hair (dyed to general population hair deposition ratios of 68%).

### General Population Hair Wet Combing Data (6 Treatment Cycles):

Wet combing was also assessed of the hair tresses after the 6 treatment cycles via a sensory panel encompassing 12 individuals.

### Wet Combing Test Method:

On the day of the last treatment cycle, the treated hair tresses were wrapped in aluminum foil and labeled in groups. During the panel, a hair tress from each leg grouping was hung on a metal bar and with each switch being detangled with the wider spacing teeth on a professional comb. The panelists then evaluated the ease of wet combing of the switches using the ‘small end’ of a professional comb (using gloved hand to stabilize switch while combing if needed) and record scores on the provided evaluation form (0-10 scale). After all 5 sets of hair have been combed (2 panelists per hair set), hang carts with hair in CT room (50% RH, 70 F).

### General Population Hair

TABLE 5
Wet Combing on General Population Hair after 6 Treatment Cycles

| | | High | |
|---|---|---|---|
| | Silicone | Melting | |
| | to High | Point Fatty | |
| | Melting Point | Compound | |
| | Fatty | to Silicone | |
| | Compound | Weight | Wet |
| Legs | Weight Ratio | Ratio | Combing |
| Clarifying Shampoo Control | — | — | 2.0 |
| Clarifying Shampoo plus | 32.5:67.5 | 67.5:32.5 | 8.2 |
| Pantene Anti-breakage | | | |
| Conditioner | | | |
| Clarifying Shampoo plus | 100:0 | 0:100 | 9.0 |
| Ex 1 Conditioner | | | |
| Clarifying Shampoo plus | 100:0 | 0:100 | 9.4 |
| Ex 2 Conditioner | | | |
| Clarifying Shampoo plus | 80:20 | 20:80 | 9.3 |
| Ex 3 Conditioner | | | |
| Clarifying Shampoo plus | 67:33 | 33:67 | 9.2 |
| Ex 4 Conditioner | | | |
| Foam Shampoo 1 plus | 100:0 | 0:100 | 8.6 |
| Ex 2 Conditioner | | | |
| Foam Shampoo 2 plus | 100:0 | 0:100 | 8.9 |
| Ex 2 Conditioner | | | |

In Table 5, the data on general population hair after 6 treatment cycles demonstrates the legs comprising the pressurized hair care compositions described herein provide good wet combing performance (from 8.6 to 9.4 average scores). But, importantly the legs comprising the pressurized hair care compositions described herein were able to do this at only ½ of the dosage (due to foams lower product density) as the liquid control regimen (0.05 grams of product per gram of hair versus 0.1 grams of product per gram of hair).

TABLE 6
Wet Combing on Dyed Hair after 6 Treatment Cycles

| | | High | |
|---|---|---|---|
| | | Melting | |
| | | Point Fatty | |
| | Silicone to High | Compound | |
| | Melting Point Fatty | to Silicone | |
| | Compound Weight | Weight | Wet |
| Regimen | Ratio | Ratio | Combing |
| Clarifying Shampoo | — | — | 1.6 |
| Control | | | |
| Clarifying Shampoo plus | 32.5:67.5 | 67.5:32.5 | 8.0 |
| Pantene Anti-breakage | | | |
| Conditioner | | | |
| Clarifying Shampoo plus | 100:0 | 0:100 | 9.3 |
| Ex 1 Conditioner | | | |
| Clarifying Shampoo plus | 100:0 | 0:100 | 9.8 |
| Ex 2 Conditioner | | | |
| Clarifying Shampoo plus | 80:20 | 20:80 | 9.4 |
| Ex 3 Conditioner | | | |
| Clarifying Shampoo plus | 67:33 | 33:67 | 9.3 |
| Ex 4 Conditioner | | | |
| Foam Shampoo 1 plus | 100:0 | 0:100 | 8.7 |
| Ex 2 Conditioner | | | |
| Foam Shampoo 2 plus | 100:0 | 0:100 | 8.6 |
| Ex 2 Conditioner | | | |

*representative of oxidatively damaged hair

In Table 6, the data on dyed hair after 6 treatment cycles demonstrates the legs comprising the pressurized hair care compositions described herein provide good wet combing performance (from 8.6 to 9.3 average scores). But, importantly the legs comprising the pressurized hair care compositions described herein were able to do this at only ½ of the dosage (due to foams lower product density) as the liquid control regimen (0.05 grams of product per gram of hair versus 0.1 grams of product per gram of hair).

### Scanning Electron Microscopy (6 Treatment Cycles)

Ten to twelve general population hair strands with 1 cm length hair from each treatment were mounted on SEM sample holder, coated with Au/Pd for 45 seconds for conductivity, transferred sample holder into SEM chamber, and used Hitachi S4700 Field Emission High Resolution SEM for imaging analysis at 3 kv with built-in Bruker Quantax Esprit SDD for EDS (Energy Dispersive X-ray Spectrometry) analysis for elemental information at 5 kv. The high-resolution image visualized the details of topography, hair structure and the deposition on its surface. EDS revealed the existence of elements of and correlated to the image topography.

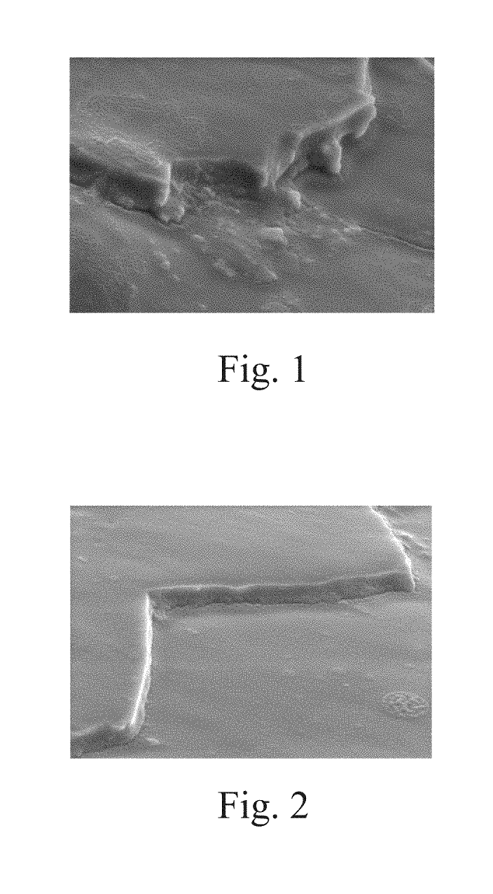

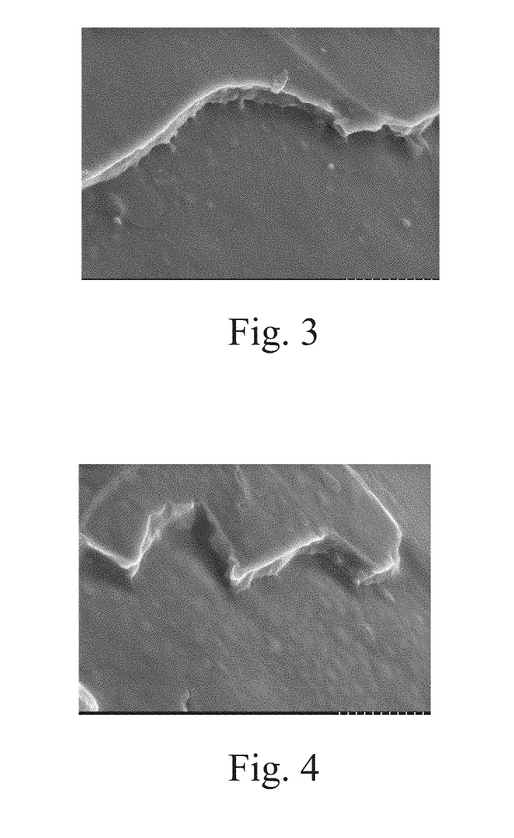

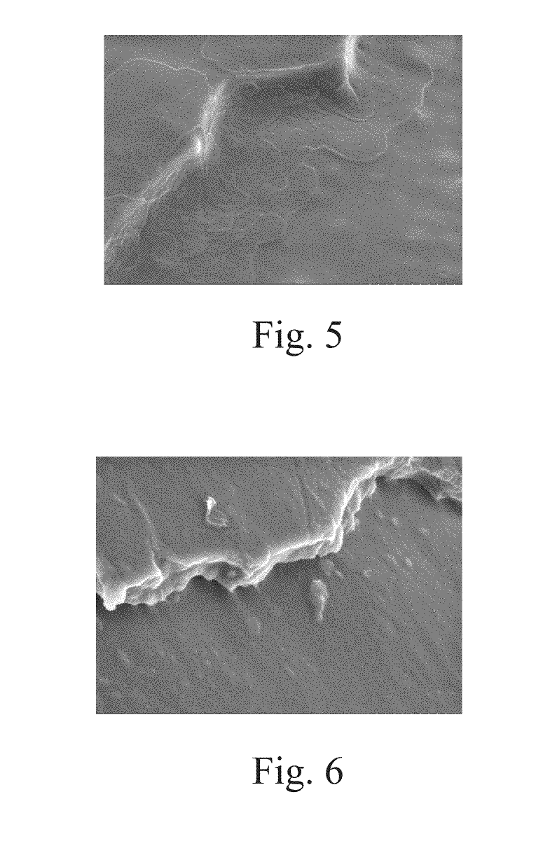

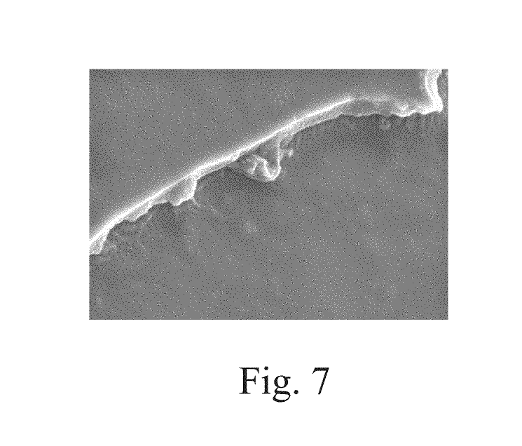

The SEM images after 6 treatment cycles on general population hair in FIGS. 1-7 show that the legs comprising the pressurized hair care compositions described herein provide surface deposits with improved morphology (thinner, smoother and more even deposition) versus the liquid control leg (irregular deposits that are thicker, not smooth and lacking even deposition). This is hypothesized to be due to significantly less co-deposits of high melting point fatty compounds (from 68% to 99% oil deposition purity) versus the liquid regimen control (from 40% to 47% oil deposition purity).

Hair/Water Contact Angle and Time of Flight SIMS (after 6 Treatment Cycles)

Hair/Water Contact Angle (General population hair): Approximately 2 cm segments from root, middle and tip were immersed in hexadecane and water root end first. The first 200 μm of the segment was ignored. Every 100 μm longitudinally up the length of the hair segment was analyzed for wetting force. Wilhelmy equation of state for rods was used to convert wetting force into contact angle. Hair diameters were measured optically.

TABLE 7
Advancing and Receding Contact Angles

| | Advancing | Receding |
|---|---|---|
| Legs | Contact Angle | Contact Angle |
| Clarifying Shampoo | 91.1 +/− 4.9 | 38.3 +/− 12.1 |
| Control | | |
| Clarifying Shampoo plus | 99.2 +/− 0.4 | 58.4 +/− 1.7 |
| Pantene Antibreakage | | |
| Conditioner | | |
| Clarifying Shampoo plus | 116.6 +/− 0.5 | 69.0 +/− 1.1 |
| Ex 1 Conditioner | | |
| Clarifying Shampoo plus | 108.9 +/− 3.2 | 70.8 +/− 0.9 |
| Ex 2 Conditioner | | |
| Clarifying Shampoo plus | 110.6 +/− 0.1 | 69.5 +/− 0.1 |
| Ex 3 Conditioner | | |
| Clarifying Shampoo plus | 108.5 +/− 0.7 | 66.8 +/− 1.9 |
| Ex 4 Conditioner | | |
| Foam Shampoo 1 plus Ex | 110.5 +/− 0.9 | 68.1 +/− 1.1 |
| 2 Conditioner | | |
| Foam Shampoo 2 plus Ex | 115.5 +/− 0.2 | 69.5 +/− 1.9 |
| 2 Conditioner | | |

The Table 7 advancing and receding contact angles after 6 treatment cycles on general population hair demonstrates that the legs comprising the pressurized hair care compositions described herein provide significantly greater hair surface hydrophobicity (advancing contact angles from 108.5 degrees to 116.6 degrees and receding contact angles from 66.8 degrees to 70.8 degrees) versus the liquid control regimen (advancing contact angle of 99.2 degrees and a receding contact angle of 58.4 degrees). This is hypothesized to be due to significantly less co-deposits of high melting point fatty compounds with the measured fatty alcohol to silicone deposition ratio being lower for the legs comprising the pressurized hair care compositions described herein (from 0 to 30:70) versus the liquid control regimen (53:47).

### Atomic Force Microscopy Test Method (6 Treatment Cycles):

Samples were prepared for AFM analysis by selecting three hairs (from treated general population hair) and adhering them to a glass microscope slide with quick curing epoxy; the analysis region was approximately the middle of the hair. AFM images were collected from one location on two fibers for each sample. Images were collected in tapping (intermittent contact) mode with a Field of View (FOV) of 40×20 mm and 512×256 pixels, yielding a spatial resolution of 78 nm. Image tilt was corrected with a first order plane fit. Force Curves were collected over the same areas imaged by AFM. Maps consisted of an array of 10 by 10 individual force curves uniformly distributed over the FOV. Adhesion values were extracted from force curves using instrument manufacturer's software. Higher magnification images were collected for a field of view of, approximately, 5×2.5 mm, yielding a resolution of 10 nm. Image tilt was corrected by a first order plain fit. Force maps were collected for several of these regions. Roughness values were obtained from height images that had been corrected for tilt (using a first order plane fit), followed by a second order plane fit to remove the hair curvature from the data. Finally another first order plane was fit to a single cuticle surface in order to remove cuticle slope from the data. (On Modify Panel, Planefit Tab, select Include Points, then draw freehand ROI on cuticle. Only the drawn ROI will be included in the mask.) Roughness was calculated over a 2.5 mm square area from six regions in each image. Step from one cuticle to next was excluded from roughness calculation; generally, it was attempted to include areas with deposition in the calculation. The AFM probe type was Olympus AC₁₆₀ (lot 9C₃₀₀₂) silicon diving board. Cantilever length is 160 nm; nominal radius for a new tip is less than 15 nm. Probes were calibrated for force measurements. One probe was used for all measurements. Three to four images were collected for each leg and the number of points analyzed was between about 500 and about 750.

TABLE 8
AFM Deposit Thickness

| | Clarifying Shampoo | Clarifying | |
|---|---|---|---|
| | plus Pantene | Shampoo | Clarifying |
| | Antibreakage | plus Ex 1 | Shampoo plus |
| | Conditioner | Conditioner | Ex 4 Conditioner |
| Avg (nm) | 32.8 | 15.4 | 15.1 |
| Std Dev (nm) | 55.0 | 9.5 | 9.8 |
| Min (nm) | 3.6 | 5.5 | 3.8 |
| Max (nm) | 439.4 | 80.2 | 100.1 |

As shown in Table 8, the legs involving the pressurized hair care compositions as described herein provide surface deposits with improved morphology (thinner, smoother and more even deposition) versus the liquid control regimen (irregular deposits that are thicker, not smooth and lacking even deposition). Additionally, the AFM was able to quantify the thickness of the deposits and demonstrate that the legs comprising the pressurized hair care compositions as described herein provide significantly thinner deposits (averages of 15.1+/−9.8 nanometers and 15.4+/−9.5 nanometers) versus the liquid control regimen (32.8+/−55 nanometers). This is hypothesized to be due to significantly less co-deposits of high melting point fatty compounds with the measured fatty alcohol deposition to silicone ratio being lower for the legs comprising the pressurized hair care compositions described herein (from 0 to 30:70) versus the liquid control regimen (53:47) It is hypothesized that the legs comprising the pressurized hair care composition described herein enable the silicone to spread better as the continuous phase of the deposit versus as the dispersed phase within a continuous phase of high melting point fatty compounds which do not spread nearly as well due to their high melting point (wax-like consistency).

### Additional Data

The Table 9 hair care compositions were prepared by weighing distilled water and the EDTA into a stainless steel beaker. The beaker was placed in a water bath on a hot plate while mixing with overhead mixer at 200 rpm and heating to 65 degrees Celsius. Cetyl alcohol and stearyl alcohol were added and the mixture and with continued heating to 80-85 C which was held there for about 10 minutes (with additional mixing speeds as needed). The behentrimonium methosulfate was then added and the mixing speed was increased to 500-600 rpm due to viscosity increase. When the materials were heated thoroughly and homogenous (about 5 to 10 minutes), the heating was stopped with continued stirring. The benzyl alcohol was then added with the batch continuing to cool to 35 C by removing the hot water from the water bath and replacing with cold water. The perfume, citric acid and Kathon were added and with continued stirring for about 10 minutes and with the formula cooling to room temperature. The silicone nano-emulsion is then added and with continued stirring at room temperature for about 10 to 15 minutes or until the formula is completely mixed and homogenous.

Propellant 1,3,3,3-hydrofluoropropene was then added to Table 9 hair care compositions at a hair care composition to propellant weight ratio of 95:5 to create pressurized hair care compositions.

TABLE 9
Hair Care Compositions before Addition of Propellant

| Raw Material | Ex. 8 | Ex. 9 |
|---|---|---|
| Amino morpholino silicone | 4 | 8 |
| (Si)⁴ | | |
| Aminosilicone (Si)² | | |
| Trideceth-5 (NE)¹ | 1.5 | 3.0 |
| C11-15 Pareth-7 (NE)² | | |
| Laureth-9 (NE)² | | |
| Trideceth-12 (NE)² | | |
| Glycerine (WMS)^{1,2} | 0.6 | 1.2 |
| Perfume (Per) | 2.0 | 2.0 |
| Behentrimonium | 4.3 | 4.3 |
| methosulfate (CS) | | |
| Cetyl Alcohol (HMPFC) | 0.86 | 0.86 |
| Stearyl Alcohol (HMPFC) | 2.15 | 2.15 |
| Citric Acid | 0.02 | 0.02 |
| Benzyl Alcohol | 0.4 | 0.4 |
| Disodium EDTA | 0.13 | 0.13 |
| Preservative (Kathon) | 0.03 | 0.03 |
| Water | (q.s.) | (q.s.) |
| Silicone Level (Si) | 4 | 8 |
| High Melting Point Fatty | 3.01 | 3.01 |
| Compound Level (HMPFC) | | |
| Cationic Surfactant Level | 4.3 | 4.3 |
| (CS) | | |
| Nonionic Emulsifiers (NE) | 1.5 | 3 |
| Perfume Level (Perf) | 2 | 2 |
| Water Miscible Solvent | 0.6 | 1.2 |
| (WMS) | | |
| Weight ratio of Silicone to | 57:43 | 73:27 |
| high melting point fatty | | |
| compounds (Si:HMPFC) | | |
| Weight ratio of high melting | 43:57 | 27:73 |
| point fatty compounds to | | |
| silicone (HMPFC:Si) | | |
| Weight ratio of Silicone to | 67:33 | 80:20 |
| Perfume (Si:Perf) | | |

¹BELSIL ® ADM 8301 E (20% active) nano-emulsion available from Wacker (<50 nm), comprising 20% amodimethicone/morpholinomethyl silsesquioxane copolymer, 5-10% trideceth-5, and 1-5% glycerin - computed average trideceth-5 emulsifier level of 7.5%; computed average glycerine level of 3%.
²CE-8170 Microemulsion (20% active) available from Dow Corning (<50 nm), comprising 20% Amodimethicone, 1-5% C11-15 Pareth-7, 1-5% Laureth-9, 1-5% Glycerin and 1-5% Trideceth-12 -- computed average C11-15 Pareth-7 emulsifier level of 3.0%; computed average laureth-9 emulsifier of 3%; computed average glycerine level of 3%; computed average trideceth-12 level of 3%.

Examples 8 and 9 from Table 9, once pressurized with the propellant, were treated onto general population hair and chemically bleached damaged hair after shampooing with Pantene Clarifying Shampoo for one treatment cycle according to the “Shampoo plus Conditioner Treatment Method” described herein. Comparisons were made to Pantene Antibreakage Conditioner which also was applied after shampooing with Pantene Clarifying Shampoo according to the “Shampoo plus Conditioner Treatment Method” described herein.

The chemically damaged hair was previously bleached with a hair bleaching composition comprising 5.5% hydrogen peroxide, 2% ammonium hydroxide, and 0.2% EDTA. The hair was submerged within this hair bleaching composition for a period of 35 minutes followed by thorough rinsing. The hair was then dried while brushing continuously for 3 minutes (1.5 minutes per side).

### General Population Hair

TABLE 10
Results of Deposition and Silicone Deposition Purity on General Population Hair after 1 Treatment Cycle

| | | | | Silicone- | Fatty | | |
|---|---|---|---|---|---|---|---|
| | Fatty | | Cationic | to-Fatty | Alcohol to | | |
| | Alcohol | Silicone | Surfactant | Alcohol | Silicone | Silicone | Total |
| | Deposition | Deposition | Deposition | Deposition | Deposition | Deposition | Deposition |
| Regimen | (ppm) | (ppm) | (ppm) | Weight Ratio | Weight Ratio | Purity | (ppm) |
| Clarifying | 1166 +/− 201 | 1049 +/− 105 | 135 +/− 31 | 47:53 | 53:47 | 47% | 2350 |
| Shampoo plus | | | | | | | |
| Pantene Anti- | | | | | | | |
| breakage | | | | | | | |
| Conditioner | | | | | | | |
| Clarifying | 473 +/− 40 | 1310 +/− 115 | 427 +/− 42 | 73:27 | 27:73 | 73% | 2210 |
| Shampoo plus | | | | | | | |
| Comparative | | | | | | | |
| Ex 14 | | | | | | | |
| Conditioner | | | | | | | |
| Clarifying | 491 +/− 24 | 1510 +/− 174 | 433 +/− 9 | 76:24 | 24:76 | 75% | 2433 |
| Shampoo plus | | | | | | | |
| Comparative | | | | | | | |
| Ex 15 | | | | | | | |
| Conditioner | | | | | | | |

In Table 10, the data demonstrates that the legs comprising the pressurized hair care compositions as described herein after 1 treatment cycle deposit higher levels of aminosilicone onto hair, importantly at only ½ of the dosage (due to foam's lower product density) as the liquid control leg (0.05 grams of product per gram of hair versus 0.1 grams of product per gram of hair) and with significantly less fatty alcohol co-deposits as compared to the control leg.

Correspondingly, the measured fatty alcohol to silicone deposition weight ratio was lower for the legs comprising the pressurized hair care compositions as described herein (27:73 and 24:76) versus the liquid control leg (53:47) Likewise, the silicone deposition purity was higher for the legs comprising the pressurized hair care compositions as described herein (73% and 75% purity) versus the liquid control leg (47% purity).

### Chemically Bleached Hair

TABLE 11
Results of Deposition and Silicone Deposition Purity on Dyed Hair after 1 Treatment Cycle

| | | | | | | | | Dyed-to- |
|---|---|---|---|---|---|---|---|---|
| | | | | Amino- | Fatty | | | General |
| | Fatty | Amino- | Cationic | silicone- | Alcohol to | | | Population |
| | Alcohol | silicone | Surfactant | to-fatty | Silicone | Oil | Total | Hair Deposition |
| | Deposition | Deposition | Deposition | alcohol | Deposition | Deposition | Deposition | Weight Ratio |
| Regimen | (ppm) | (ppm) | (ppm) | ratio | Weight Ratio | Purity | (ppm) | (×100%) |
| Clarifying | 455 +/− 24 | 294 +/− 44 | 89 +/− 8 | 39:61 | 61:39 | 39% | 839 | 28% |
| Shampoo plus | | | | | | | | |
| Pantene Anti- | | | | | | | | |
| breakage | | | | | | | | |
| Conditioner | | | | | | | | |
| Clarifying | 482 +/− 47 | 1070 +/− 61 | 482 +/− 52 | 69:31 | 31:69 | 69% | 2034 | 82% |
| Shampoo plus | | | | | | | | |
| Comparative Ex | | | | | | | | |
| 14 Conditioner | | | | | | | | |
| Clarifying | 434 +/− 24 | 1253 +/− 70 | 503 +/− 5 | 74:26 | 26:74 | 74% | 2190 | 83% |
| Shampoo plus | | | | | | | | |
| Comparative Ex | | | | | | | | |
| 15 Conditioner | | | | | | | | |

In Table 11, the data demonstrates that the legs comprising the pressurized hair care compositions as described herein after 1 treatment cycle deposit significantly higher levels of aminosilicone onto chemically bleached damaged hair at only ½ of the dosage (due to foam's lower product density) as the liquid control leg (0.05 grams of product per gram of hair versus 0.1 grams of product per gram of hair). Correspondingly, the measured fatty alcohol to silicone deposition weight ratio was lower for the legs comprising the pressurized hair care compositions as described herein (31:69 and 26:74) versus the liquid control leg (61:39). Likewise, the silicone deposition purity was much higher for the legs comprising the pressurized hair care compositions as described herein (69% and 74% purity) versus the liquid control leg (39% purity). Moreover, the legs comprising the pressurized hair care compositions as described herein also deposit a significantly greater amount of silicone onto the more chemically bleached damaged hair than on general population hair (dyed to general population hair deposition weight ratios of 82% and 83%) versus the liquid control leg which deposited less silicone on the more polar chemically bleached hair (dyed to general population hair deposition weight ratio of 28%).

### ADDITIONAL EXAMPLES

The following hair care compositions can be prepared by the following method:

Begin by weighing distilled water and the EDTA into a stainless steel beaker. The beaker is placed in a water bath on a hot plate while mixing with overhead mixer at 200 rpm and heating to 65 degrees Celsius. The fatty alcohol is added to the mixture with continued heating to 80-85C which is held there for about 10 minutes (with additional mixing speeds as needed). The cationic surfactant is then added and the mixing speed is increased to 500-600 rpm due to viscosity increase. When the materials are heated thoroughly and homogenous (about 5 to 10 minutes), the heating is stopped with continued stirring. The benzyl alcohol is then added with the batch continuing to cool to 35 C by removing the hot water from the water bath and replacing with cold water. The perfume, citric acid and Kathon are added with continued stirring for about 10 minutes as the formula cools to room temperature. The silicone nano-emulsion is then added with continued stirring at room temperature for about 10 to 15 minutes or until the formula is completely mixed and homogenous.

To create a pressurized hair care composition, a propellant can be added to the hair care composition at a hair care composition to propellant weight ratio of from about 85:15 to about 98:2; alternatively from about 90:10 to about 97:3; and alternatively from about 92:8 to about 96:4 to create a pressurized hair care composition. The propellant can be added to the hair care composition within an aerosol dispenser or it can be pre-mixed prior to injection into the aerosol dispenser.

TABLE 12
Additional Hair Care Compositions before Addition of Propellant

| Raw Material | Ex 10 | Ex 11 | Ex 12 | Ex 13 | Ex 14 | Ex 15 |
|---|---|---|---|---|---|---|
| Aminosilicone (Si)¹ | 8 | 4 | 2 | 0 | 8 | 4 |
| Aminosilicone (Si)² | | 4 | 2 | 4 | | 4 |
| Alcohol ethoxylates (NE)¹ | 5.0 | 2.5 | 1.25 | | 5.0 | 2.5 |
| Glycerine (WMS)¹ | 1.2 | 0.6 | 0.3 | | 1.2 | 0.6 |
| Perfume | 2.0 | 2.0 | 2.0 | 2.0 | 2.0 | 2.0 |
| Behentrimonium | 4.3 | 4.3 | 4.3 | 4.3 | 4.3 | 4.3 |
| methosulfate (CS) | | | | | | |
| Cetyl Alcohol (HMPFC) | 0.86 | 0.86 | 0.86 | 0.86 | 0.86 | 0.86 |
| Stearyl Alcohol (HMPFC) | 2.15 | 2.15 | 2.15 | 2.15 | 2.15 | 2.15 |
| Hydroxyethyl cellulose³ | 0.00 | 0.00 | 0.00 | 0.00 | 1.00 | 0.5 |
| Citric Acid | 0.02 | 0.02 | 0.02 | 0.02 | 0.02 | 0.02 |
| Benzyl Alcohol | 0.4 | 0.4 | 0.4 | 0.4 | 0.4 | 0.4 |
| Disodium EDTA | 0.13 | 0.13 | 0.13 | 0.13 | 0.13 | 0.13 |
| Preservative (Kathon) | 0.03 | 0.03 | 0.03 | 0.03 | 0.03 | 0.03 |
| Water | (q.s.) | (q.s.) | (q.s.) | (q.s.) | (q.s.) | (q.s.) |
| Silicone Level (Si) | 8 | 8 | 4 | 4 | 8 | 8 |
| High Melting Point Fatty | 0 | 0 | 3.0 | 6.0 | 8.0 | 12.0 |
| Compound Level (HMPFC) | | | | | | |
| Cationic Surfactant Level | 0 | 2.5 | 2.5 | 2.5 | 2.5 | 2.5 |
| (CS) | | | | | | |
| Nonionic Emulsifiers (NE) | 7.5 | 7.5 | 7.5 | 7.5 | 7.5 | 7.5 |
| Perfume Level | 2.0 | 2.0 | 2.0 | 2.0 | 2.0 | 2.0 |
| Water Miscible Solvent | 1.2 | 0.6 | 0.3 | | 1.2 | 0.6 |
| (WMS) | | | | | | |
| Weight ratio of Silicone to | 73:27 | 73:27 | 57:43 | 57:43 | 73:27 | 73:27 |
| high melting point fatty | | | | | | |
| compounds (Si:HMPFC) | | | | | | |
| Weight ratio of high melting | 27:73 | 27:73 | 43:57 | 43:57 | 27:73 | 27:73 |
| point fatty compounds to | | | | | | |
| silicone (HMPFC:Si) | | | | | | |
| Weight ratio of Silicone to | 80:20 | 80:20 | 67:33 | 67:33 | 80:20 | 80:20 |
| Perfume (Si:Perf) | | | | | | |
| Viscosity (cp) | 591 | 756 | 1461 | 9505 | 6830 | 3202 |

¹Silsoft 253 (20% active) nano-emulsion available from Momentive (10-20 nm), comprising secondary alcohol ethoxylate (5-10%), C12-16 synthetic alcohols-ethoxylated (1-5%), glycerine (1-5%), and tridecyl alcohol ethoxylate (1-5%) - computed average alcohol ethoxylates emulsifier level of 12.5%; computed average glycerine level of 3%.
²Y17045 (100% active) available experimentally from Momentive
³Natrosol 250HHR available from Ashland Chemicals.

TABLE 13
Additional Hair Care Compositions before Addition of Propellant

| Raw Material | Ex 16 | Ex 17 | Ex 18 | Ex 19 | Ex 20 |
|---|---|---|---|---|---|
| Amino morpholino silicone | 12 | 3.5 | | | 3 |
| (Si)⁴ | | | | | |
| Aminosilicone (Si)⁵ | | | 8 | 12 | |
| Trideceth-5 (NE)⁴ | 4.5 | 1.3 | | | 1.1 |
| C11-15 Pareth-7 (NE)⁵ | | | 1.2 | 1.8 | |
| Laureth-9 (NE)⁵ | | | 1.2 | 1.8 | |
| Trideceth-12 (NE)⁵ | | | 1.2 | 1.8 | |
| Glycerine (WMS)^{4,5} | 1.8 | 0.5 | 1.2 | 1.8 | 0.5 |
| Perfume (Per) | 2.0 | 2.0 | 2.0 | 2.0 | 1.5 |
| Behentrimonium | 4.3 | 4.3 | 4.3 | 4.3 | 4.3 |
| methosulfate (CS) | | | | | |
| Cetyl Alcohol (HMPFC) | 0.86 | 0.86 | 0.86 | 0.86 | 0.86 |
| Stearyl Alcohol (HMPFC) | 2.15 | 2.15 | 2.15 | 2.15 | 2.15 |
| Citric Acid | 0.02 | 0.02 | 0.02 | 0.02 | 0.02 |
| Benzyl Alcohol | 0.4 | 0.4 | 0.4 | 0.4 | 0.4 |
| Disodium EDTA | 0.13 | 0.13 | 0.13 | 0.13 | 0.13 |
| Preservative (Kathon) | 0.03 | 0.03 | 0.03 | 0.03 | 0.03 |
| Water | (q.s.) | (q.s.) | (q.s.) | (q.s.) | (q.s.) |
| Silicone Level (Si) | 12 | 3.5 | 8 | 12 | 3 |
| High Melting Point Fatty | 3.01 | 3.01 | 3.01 | 3.01 | 3.01 |
| Compound Level (HMPFC) | | | | | |
| Cationic Surfactant Level | 4.3 | 4.3 | 4.3 | 4.3 | 4.3 |
| (CS) | | | | | |
| Nonionic Emulsifiers (NE) | 4.5 | 1.3 | 3.6 | 5.4 | 1.1 |
| Perfume Level (Perf) | 2 | 2 | 2 | 2 | 1.5 |
| Water Miscible Solvent | 1.8 | 0.5 | 1.2 | 1.8 | 0.5 |
| (WMS) | | | | | |
| Weight ratio of Silicone to | 80:20 | 54:46 | 73:27 | 80:20 | 50:50 |
| high melting point fatty | | | | | |
| compounds (Si:HMPFC) | | | | | |
| Weight ratio of high melting | 20:80 | 46:54 | 27:73 | 20:80 | 50:50 |
| point fatty compounds to | | | | | |
| silicone (HMPFC:Si) | | | | | |
| Weight ratio of Silicone to | 86:14 | 64:36 | 80:20 | 86:14 | 67:33 |
| Perfume (Si:Perf) | | | | | |

⁴BELSIL ® ADM 8301 E (20% active) nano-emulsion available from Wacker (<50 nm), comprising 20% amodimethicone/morpholinomethyl silsesquioxane copolymer, 5-10% trideceth-5, and 1-5% glycerin - computed average trideceth-5 emulsifier level of 7.5%; computed average glycerine level of 3%.
⁵CE-8170 Microemulsion (20% active) available from Dow Corning (<50 nm), comprising 20% Amodimethicone, 1-5% C11-15 Pareth-7, 1-5% Laureth-9, 1-5% Glycerin and 1-5% Trideceth-12 -- computed average C11-15 Pareth-7 emulsifier level of 3.0%; computed average laureth-9 emulsifier of 3%; computed average glycerine level of 3%; computed average trideceth-12 level of 3%.

TABLE 14
Additional Hair Care Compositions before Addition of Propellant

| Raw Material | Ex 21 | Ex 22 | Ex 23 | Ex 24 | Ex 25 |
|---|---|---|---|---|---|
| Amino morpholino silicone (Si)⁴ | 4 | 8 | 8 | 8 | 8 |
| Aminosilicone (Si)⁵ | | | | | |
| Trideceth-5 (NE)⁴ | 1.50 | 3.00 | | 3.00 | 3.00 |
| C11-15 Pareth-7 (NE)⁵ | | | 1.2 | | |
| Laureth-9 (NE)⁵ | | | 1.2 | | |
| Trideceth-12 (NE)⁵ | | | 1.2 | | |
| Glycerin (WMS)^{4,5} | 0.60 | 1.20 | 1.80 | 1.20 | 1.20 |
| Perfume (Per) | 2.00 | 2.00 | 4.00 | 5.00 | 5.00 |
| Behentrimonium methosulfate (CS) | 4.30 | 4.30 | 4.30 | 4.30 | 4.30 |
| Cetyl Alcohol (HMPFC) | 0.86 | 0.86 | 0.86 | 0.86 | 3.01 |
| Stearyl Alcohol (HMPFC) | 2.15 | 2.15 | 2.15 | 2.15 | |
| Citric Acid | 0.02 | 0.02 | 0.02 | 0.02 | 0.02 |
| Benzyl Alcohol | 0.40 | 0.40 | 0.40 | 0.40 | 0.40 |
| Disodium EDTA | 0.13 | 0.13 | 0.13 | 0.13 | 0.13 |
| Preservative (Kathon) | 0.033 | 0.033 | 0.033 | 0.033 | 0.033 |
| Water | (q.s.) | (q.s.) | (q.s.) | (q.s.) | (q.s.) |
| Silicone Level (Si) | 4.00 | 8.00 | 8.00 | 8.00 | 8.00 |
| High Melting Point Fatty | 3.01 | 3.01 | 3.01 | 3.01 | 3.01 |
| Compound Level (HMPFC) | | | | | |
| Cationic Surfactant Level (CS) | 4.3 | 4.30 | 4.30 | 4.30 | 4.30 |
| Nonionic Emulsifiers (NE) | 1.5 | 3.00 | 3.60 | 3.00 | 3.00 |
| Perfume Level (Perf) | 2 | 2.00 | 4.00 | 5.00 | 5.00 |
| Water Miscible Solvent (WMS) | 0.6 | 1.20 | 1.80 | 1.20 | 1.20 |
| Weight ratio of Silicone to high | 57:43 | 73:27 | 73:27 | 73:27 | 73:27 |
| melting point fatty compounds | | | | | |
| (Si:HMPFC) | | | | | |
| Weight ratio of high melting point | 43:57 | 27:73 | 27:73 | 27:73 | 27:73 |
| fatty compounds to silicone | | | | | |
| (HMPFC:silicone) | | | | | |
| Weight ratio of Silicone to Perfume | 67:33 | 80:20 | 67:33 | 62:38 | 62:38 |
| (Si:Perf) | | | | | |

⁴BELSIL ® ADM 8301 E (20% active) nano-emulsion available from Wacker (<50 nm), comprising 20% amodimethicone/morpholinomethyl silsesquioxane copolymer, 5-10% trideceth-5, and 1-5% glycerin - computed average trideceth-5 emulsifier level of 7.5%; computed average glycerine level of 3%.
⁵CE-8170 Microemulsion (20% active) available from Dow Corning (<50 nm), comprising 20% Amodimethicone, 1-5% C11-15 Pareth-7, 1-5% Laureth-9, 1-5% Glycerin and 1-5% Trideceth-12 -- computed average C11-15 Pareth-7 emulsifier level of 3.0%; computed average laureth-9 emulsifier of 3%; computed average glycerine level of 3%; computed average trideceth-12 level of 3%.

TABLE 15
Additional Hair Care Compositions before Addition of Propellant

| Raw Material | Ex 26 | Ex. 27 | Ex 28 | Ex 29 | Ex 30 |
|---|---|---|---|---|---|
| Amino morpholino silicone (Si)⁴ | 8.00 | 8.00 | 8.00 | 8.00 | 8.00 |
| Aminosilicone (Si)⁵ | | | | | |
| Trideceth-5 (NE)⁴ | 3.00 | 3.00 | 3.00 | 3.00 | 3.00 |
| C11-15 Pareth-7 (NE)⁵ | | | | | |
| Laureth-9 (NE)⁵ | | | | | |
| Trideceth-12 (NE)⁵ | | | | | |
| Glycerin (WMS)^{4,5} | 1.20 | 1.20 | 1.20 | 1.20 | 1.20 |
| Perfume (Per) | 5.00 | 5.00 | 3.00 | 3.00 | 3.00 |
| Behentrimonium methosulfate (CS) | 4.30 | 4.30 | 2.10 | 1.00 | 0.50 |
| Cetyl Alcohol (HMPFC) | | 0.86 | 0.43 | 0.22 | 0.11 |
| Stearyl Alcohol (HMPFC) | | | 1.07 | 0.54 | 0.27 |
| Behenyl Alcohol (HMPFC) | 3.01 | 2.15 | | | |
| Citric Acid | 0.02 | 0.02 | 0.02 | 0.02 | 0.02 |
| Benzyl Alcohol | 0.40 | 0.40 | 0.40 | 0.40 | 0.40 |
| Disodium EDTA | 0.13 | 0.13 | 0.13 | 0.13 | 0.13 |
| Preservative (Kathon) | 0.033 | 0.033 | 0.033 | 0.033 | 0.033 |
| Water | (q.s.) | (q.s.) | (q.s.) | (q.s.) | (q.s.) |
| Silicone Level (Si) | 8.00 | 8.00 | 8.00 | 8.00 | 8.00 |
| High Melting Point Fatty | 3.01 | 3.01 | 1.50 | 0.76 | 0.38 |
| Compound Level (HMPFC) | | | | | |
| Cationic Surfactant Level (CS) | 4.30 | 4.30 | 2.10 | 1.00 | 0.50 |
| Nonionic Emulsifiers (NE) | 3.00 | 3.00 | 3.00 | 3.00 | 3.00 |
| Perfume Level (Perf) | 5.00 | 5.00 | 3.00 | 3.00 | 3.00 |
| Water Miscible Solvent (WMS) | 1.20 | 1.20 | 1.20 | 1.20 | 1.20 |
| Weight ratio of Silicone to high | 73:27 | 73:27 | 84:16 | 91:9 | 95:5 |
| melting point fatty compounds | | | | | |
| (Si:HMPFC) | | | | | |
| Weight ratio of high melting point | 27:73 | 27:73 | 16:84 | 9:91 | 5:95 |
| fatty compounds to silicone | | | | | |
| (HMPFC:Si) | | | | | |
| Weight ratio of Silicone to | 62:38 | 62:38 | 73:27 | 73:27 | 73:27 |
| Perfume (Si:Perf) | | | | | |

⁴BELSIL ® ADM 8301 E (20% active) nano-emulsion available from Wacker (<50 nm), comprising 20% amodimethicone/morpholinomethyl silsesquioxane copolymer, 5-10% trideceth-5, and 1-5% glycerin - computed average trideceth-5 emulsifier level of 7.5%; computed average glycerine level of 3%.
⁵CE-8170 Microemulsion (20% active) available from Dow Corning (<50 nm), comprising 20% Amodimethicone, 1-5% C11-15 Pareth-7, 1-5% Laureth-9, 1-5% Glycerin and 1-5% Trideceth-12 -- computed average C11-15 Pareth-7 emulsifier level of 3.0%; computed average laureth-9 emulsifier of 3%; computed average glycerine level of 3%; computed average trideceth-12 level of 3%.

### Additional Combinations:

A. An aerosol dispenser comprising a pressurized personal care product in a bag on valve container, the pressurized personal care product comprising:
   a) from about 2% to about 10% propellant;
   b) from about 0.5% to about 5% perfume;
   c) from about 1% to about 14% nonionic emulsifier;
   d) less than 5% cationic surfactant;
   e) from about 55% to about 87% water;
   f) from about 3% to about 17% silicone, wherein the particle size of the one or more silicones is from about 1 nm to about 500 nm;
   g) less than 7.5% high melting point fatty compound;
      wherein the pressurized composition has a liquid phase viscosity of from about 1 centipoise to about 15,000 centipoise;
      wherein the pressurized composition has a silicone to high melting point fatty compound weight ratio of from about 100:0 to about 50:50;
      wherein the pressurized composition has a silicone to perfume weight ratio of from about 95:5 to about 50:50;
      wherein the aerosol dispenser dispenses a foam having a density of from about 0.025 g/cm³ to about 0.40 g/cm³ when dispensed from the aerosol dispenser; and wherein the pressurized composition is rinse-off.
B. The aerosol dispenser of paragraph A, wherein the pressurized hair care composition comprises from about 62% to about 85% water.
C. The aerosol dispenser of paragraph A or paragraph B, wherein the pressurized hair care composition has a liquid phase viscosity of from about 1 centipoise to about 8,000 centipoise.
D. The aerosol dispenser of any preceding paragraph in this section, wherein the silicone is selected from the group consisting of aminosilicones, pendant quaternary ammonium silicones, terminal quaternary ammonium silicones, amino polyalkylene oxide silicones, quaternary ammonium polyalkylene oxide silicones, amino morpholino silicones, and mixtures thereof.
E. The aerosol dispenser of any preceding paragraph in this section, wherein the pressurized hair care composition comprises from about 5% to about 15% of one or more silicones, by weight of the pressurized hair care composition.
F. The aerosol dispenser of any preceding paragraph in this section, wherein the pressurized hair care composition comprises from about 2% to about 9.5% of a nonionic emulsifier, by weight of the pressurized hair care composition.
G. The aerosol dispenser of any preceding paragraph in this section, wherein the nonionic emulsifier is a condensation product of an aliphatic alcohol having from about 8 to about 18 carbons, in either straight chain or branched chain configuration, with from about 2 to about 35 moles of ethylene oxide.
H. The aerosol dispenser of any preceding paragraph in this section, wherein the foam comprises a silicone to fatty alcohol deposition weight ratio of from about 50:50 to about 100:0.
I. The aerosol dispenser of any preceding paragraph in this section, wherein the foam comprises a silicone to fatty alcohol deposition weight ratio of from about 60:40 to about 100:0.
J. The aerosol dispenser of any preceding paragraph in this section, wherein the foam comprises a silicone deposition purity of from about 50% to about 100%.
K. The aerosol dispenser of any preceding paragraph in this section, wherein the foam comprises a silicone deposition purity of from about 60% to about 100%.
L. The aerosol dispenser of any preceding paragraph in this section, wherein the particle size of the silicone is from about 5 nm to about 300 nm.
M. The aerosol dispenser of any preceding paragraph in this section, wherein the pressurized hair care composition is in the form of a nanoemulsion.
N. The aerosol dispenser of any preceding paragraph in this section, wherein from about 25% to about 100% of the silicone is in the form of a nanoemulsion, by weight of the pressurized hair care composition.
O. The aerosol dispenser of any preceding paragraph in this section, wherein the pressurized hair care composition is substantially free of high melting point fatty compounds, by weight of the pressurized hair care composition.
P. The aerosol dispenser of any preceding paragraph in this section, wherein the pressurized hair care composition comprises less than 4% high melting point fatty compounds, by weight of the pressurized hair care composition.
Q. The aerosol dispenser of any preceding paragraph in this section, wherein the pressurized hair care composition comprises from about 1.25% to about 4.0% perfume, by weight of the pressurized hair care composition.
R. The aerosol dispenser of any preceding paragraph in this section, wherein the foam has a dosage weight of from about 1 g to about 5 g when dispensed from the aerosol dispenser.
S. The aerosol dispenser of any preceding paragraph in this section, wherein the density of the foam is from about 0.035 g/cm³ to about 0.20 g/cm³.
T. The aerosol dispenser of any preceding paragraph in this section, wherein the liquid phase viscosity of the pressurized hair care composition is from about 10 centipoise to about 2,500 centipoise.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) An aerosol dispenser wherein the aerosol dispenser is a bag on valve system comprising an inner bag and an outer container wherein the inner bag comprises a a pressurized personal care product, the pressurized personal care product comprising:
a) from about 2% to about 10% propellant;
b) from about 0.5% to about 5% perfume;
c) from about 1% to about 14% nonionic emulsifier;
d) less than 5% cationic surfactant;
e) from about 55% to about 87% water;
f) from about 3% to about 17% silicone, wherein the particle size of the one or more silicones is from about 1 nm to about 500 nm;
g) less than 7.5% high melting point fatty compound;
wherein the pressurized composition has a liquid phase viscosity of from about 1 centipoise to about 15,000 centipoise;
wherein the pressurized composition has a high melting point fatty compound to silicone weight ratio of from about 0 to about 50:50;
wherein the pressurized composition has a silicone to perfume weight ratio of from about 50:50 to about 95:5;
wherein the aerosol dispenser dispenses a foam having a density of from about 0.025 g/cm³ to about 0.40 g/cm³ when dispensed from the aerosol dispenser; and
wherein the pressurized composition is rinse-off.

2) The aerosol dispenser of claim 1, wherein the pressurized hair care composition comprises from about 62% to about 85% water.

3) The aerosol dispenser of claim 1, wherein the pressurized hair care composition has a liquid phase viscosity of from about 1 centipoise to about 8,000 centipoise.

4) The aerosol dispenser of claim 1, wherein the silicone is selected from the group consisting of aminosilicones, pendant quaternary ammonium silicones, terminal quaternary ammonium silicones, amino polyalkylene oxide silicones, quaternary ammonium polyalkylene oxide silicones, amino morpholino silicones, and mixtures thereof.

5) The aerosol dispenser of claim 1, wherein the pressurized hair care composition comprises from about 4% to about 15% of one or more silicones, by weight of the pressurized hair care composition.

6) The aerosol dispenser of claim 1, wherein the pressurized hair care composition comprises from about 2% to about 9.5% of a nonionic emulsifier, by weight of the pressurized hair care composition.

7) The aerosol dispenser of claim 1, wherein the nonionic emulsifier is a condensation product of an aliphatic alcohol having from about 8 to about 18 carbons, in either straight chain or branched chain configuration, with from about 2 to about 35 moles of ethylene oxide.

8) The aerosol dispenser of claim 1, wherein the high melting point fatty compound comprises a fatty alcohol and wherein the foam comprises a fatty alcohol to silicone deposition weight ratio of from about 0 to about 50:50.

9) The aerosol dispenser of claim 8, wherein the foam comprises a fatty alcohol to silicone deposition weight ratio of from about 0 to about 60:40.

10) The aerosol dispenser of claim 1, wherein the foam comprises a silicone deposition purity of from about 50% to about 100%.

11) The aerosol dispenser of claim 1, wherein the foam comprises a silicone deposition purity of from about 60% to about 100%.

12) The aerosol dispenser of claim 1, wherein the particle size of the silicone is from about 5 nm to about 300 nm.

13) The aerosol dispenser of claim 1, wherein the pressurized hair care composition is in the form of a nanoemulsion.

14) The aerosol dispenser of claim 1, wherein from about 25% to about 100% of the silicone is in the form of a nanoemulsion, by weight of the pressurized hair care composition.

15) The aerosol dispenser of claim 1, wherein the pressurized hair care composition is substantially free of high melting point fatty compounds, by weight of the pressurized hair care composition.

16) The aerosol dispenser of claim 1, wherein the pressurized hair care composition comprises less than 4% high melting point fatty compounds, by weight of the pressurized hair care composition.

17) The aerosol dispenser of claim 1, wherein the pressurized hair care composition comprises from about 1.25% to about 4.0% perfume, by weight of the pressurized hair care composition.

18) The aerosol dispenser of claim 1, wherein the foam has a dosage weight of from about 1 g to about 5 g when dispensed from the aerosol dispenser.

19) The aerosol dispenser of claim 1, wherein the density of the foam is from about 0.035 g/cm³ to about 0.20 g/cm³.

20) The aerosol dispenser of claim 1, wherein the liquid phase viscosity of the pressurized hair care composition is from about 10 centipoise to about 2,500 centipoise.
